(19) United States
(12) Patent Application Publication
(10) Pub. No.: US 2011/0003880 A1
(43) Pub. Date: Jan. 6, 2011

(54) Compositions and methods for inhibiting optic nerve damage

(76) Inventors: Sanjoy K. Bhattacharya, Miami, FL (US); John W. Crabb, Chagrin Falls, OH (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/228,429

(22) Filed: Aug. 12, 2008

Related U.S. Application Data
(63) Continuation of application No. PCT/US2007/003834, filed on Feb. 12, 2007.
(60) Provisional application No. 60/773,359, filed on Feb. 13, 2006.

(51) Int. Cl. A61K 31/713; C12Q 1/37; A61P 27/06; A61P 27/02; G01N 33/00

(52) U.S. Cl. 514/44 A; 435/24; 800/9

(57) ABSTRACT

Provided herein is a method of inhibiting optic nerve damage in an individual in need thereof, comprising administering to the individual an agent that inhibits peptidyl arginine deiminase 2 (PAD2). In a particular embodiment, the present invention is directed to a method of inhibiting glaucomatous optic nerve damage in an individual in need thereof, comprising administering to the individual an agent that inhibits peptidyl arginine deiminase 2 (PAD2). The present invention is also directed to a method of treating glaucoma (e.g., primary open angle glaucoma) in an individual in need thereof, comprising administering to the individual an agent that inhibits (e.g., specifically inhibits) peptidyl arginine deiminase 2 (PAD2).

### RELATED APPLICATIONS

This application is a continuation of International Application No. PCT/US2007/003834, which designated the United States and was filed on Feb. 12, 2007, published in English, which claims the benefit of U.S. Provisional Application No. 60/773,359, filed on Feb. 13, 2006. The entire teachings of the above applications are incorporated herein by reference.

### GOVERNMENT SUPPORT

The invention was supported, in whole or in part, by grants EY015266, EY06603, EY014239 and EY015638 from the National Institutes of Health. The Government has certain rights in the invention.

### BACKGROUND OF THE INVENTION

Glaucoma is a group of poorly understood neurodegenerative disorders characterized by deformation of the optic nerve head (ONH), loss of retinal ganglion cells and irreversible vision loss in about 70 million people worldwide (Quigley, H. A., Br. J Ophthalmol. (1996) 80:389-93). The risk of glaucoma increases with age, with the disease at age 80 being 5 to 10 times more prevalent than at age 40 (Gordon, M. O., et al., Arch Ophthalmol. (2002) 120:714-20). Glaucomas are classified as primary when they occur with no known etiology, or as secondary, where a previous illness or injury is contributory. In primary open angle glaucoma (POAG) most but not all patients exhibit elevated intraocular pressure (IOP) which leads to optic nerve damage, often termed glaucomatous optic neuropathy (Ahmed, F., et al., Invest Ophthalmol Vis Sci. (2001) 42:3165-72).

A need exists for improved methods of diagnosing and treating glaucoma.

### SUMMARY OF THE INVENTION

Described herein is proteomic analyses of normal and glaucomatous optic nerve, which showed increased levels of peptidyl arginine deiminase 2 (protein deiminase 2 or PAD2) in glaucomatous, but not in normal, optic nerve. Glaucomas are divided into two main categories: primary, where no apparent cause for onset can be attributed, and secondary, where an apparent cause such as previous injury or illness can be identified. Primary glaucoma is further divided into two groups, open angle (POAG), and angle-closure (PACG). POAG is the most common form of the disease, glaucoma affects about 3 million Americans and more than 70 million people worldwide (Thylefors, B., et al., Bull. World Health Organ., 73:115-121 (1995); Quigley, H. A., et al., Br. J. Ophthalmol., 80:389-393 (1996)). PAD2 enzyme activity is modulated by calcium and converts protein arginine to citrulline (Vossenaar, E. R., et al., Bioessays, 25:1106-1118 (2003)). It was also found that POAG optic nerve exhibits increased citrullination and several citrullinated optic nerve proteins, including myelin basic protein, have been identified. Concomitant with increased citrullination in POAG optic nerve, decreased protein arginyl methylation was observed, indicating that structural disruption of myelination likely contributes to optic nerve degeneration in POAG. Also provided herein is in vitro evidence of pressure-induced translational control of PAD2 expression, consistent with a role for PAD2 and citrullination in POAG pathology.

Accordingly, the present invention provides for methods of treating and/or diagnosing optic nerve damage and glaucoma. In particular, the invention is directed to a method of inhibiting (e.g., directly, indirectly) optic nerve damage in an individual in need thereof, comprising administering to the individual an agent that inhibits peptidyl arginine deiminase 2 (PAD2). The agent can inhibit expression of PAD2, biological activity of PAD2 (e.g., increased protein citrullination, decreased protein arginyl methylation) or a combination thereof. In a particular embodiment, the agent directly inhibits the expression and/or biological activity of PAD2 (e.g., an antibody that specifically binds PAD2; PAD2 interfering RNA). In a particular embodiment, the present invention is also directed to a method of inhibiting glaucomatous optic nerve damage in an individual in need thereof, comprising administering to the individual an agent that inhibits peptidyl arginine deiminase 2 (PAD2).

The present invention is also directed to a method of treating glaucoma (e.g., primary open angle glaucoma) in an individual in need thereof, comprising administering to the individual an agent that inhibits (e.g., specifically inhibits) peptidyl arginine deiminase 2 (PAD2).

In addition, methods of screening for agents (compounds) that can be used to treat and/or inhibit optic nerve damage (e.g., glaucoma) are provided. Thus, a method of identifying an agent that can be used to inhibit optic nerve damage is also encompassed by the invention. The method comprises contacting a cell (e.g., an ocular cell such as an astrocyte) or animal (e.g., an animal model of glaucoma such as the DBA/2J mouse model) which expresses peptidyl arginine deiminase 2 (PAD2) with an agent to be assessed. The level of expression or biological activity of PAD2 in the cell of animal is assessed, wherein if the level of expression or biological activity of PAD2 is decreased in the presence of the agent, then the agent can be used to inhibit optic nerve damage. In one embodiment, the biological activity of PAD2 that is assessed is citrullination and if citrullination is increased, then the agent can be used to inhibit optic nerve damage (e.g., optic nerve damage associate with glaucoma).

### BRIEF DESCRIPTION OF THE DRAWINGS

FIGS. 1A-1D. Elevated PAD2 levels and citrullination in glaucomatous optic nerve. FIG. 1A, Representative SDS-PAGE of human optic nerve protein (˜10 μg per lane) from POAG and control donors (Coomassie blue staining). Gel slices were excised and proteins identified by LC MS/MS (see Table 1). FIG. 1B, Representative Western analyses with monoclonal anti-PAD2 of protein extracts from human optic nerve demonstrating the presence of ˜72 KDa protein uniquely in glaucomatous tissues. FIG. 1C, Western analyses with rabbit polyclonal antibody to citrulline (10 μg protein per lane). Prior to applying antibody, membrane immobilized protein was treated with 2,3-butanedione monooxime and antipyrine in a strong acid atmosphere enabling chemical modification of citrulline into ureido groups and ensured detection of citrulline-containing proteins regardless of neighboring amino acid sequences. FIG. 1D, Western analyses with mouse monoclonal antibody to protein methylarginine (10 μg protein per lane). Protein was extracted from the optic nerve of Caucasian cadaver donor eyes: age and gender are indicated.

FIGS. 2A-2B. Western analysis using PAD2 and citrulline antibodies. FIG. 2A, Anti-PAD2 Western analyses of Control (C57BL6J) and DBA/2J mice optic at indicated ages (in months). FIG. 2B, Anti-Citrulline Western analyses of mice optic nerve.

FIGS. 3A-3E. Immunohistochemical Localization of PAD2 and citrullinated proteins in the optic nerve. Control and glaucomatous optic nerve scanning confocal microscopic images are shown on the top and bottom rows, respectively, with the age and gender of the Caucasian tissue donors. FIGS. 3A, 3B, anti-PAD2 staining (secondary conjugated with Alexa 594) images; PAD2 immunoreactivity is predominantly observed in glaucomatous optic nerve. FIGS. 3C, 3D, Control and glaucomatous optic nerve images stained with anti-citrulline antibodies (secondary conjugated with Alexa 488). Citrulline immunoreactivity is predominantly observed in glaucomatous optic nerve. FIG. 3E, The optic nerve; in particular, the dissected region of the optic nerve (lamina cribrosa) and the DAPI stained fluoresence microscope image is shown. (Illustrated by S. K. Bhattacharya, Cole Eye Institute and D. Schumick, Department of Medical Illustration, Cleveland Clinic Foundation. ©2005, Cleveland Clinic Foundation.)

FIGS. 4A-4E. Immunoprecipitation of Human Optic Nerve Proteins. FIG. 4A, Commassie blue detection of immunoprecipitation (IP) products from glaucomatous (G) and normal (N) optic nerve proteins with anti-citrulline or anti-myelin basic protein (MBP). FIG. 4B, Commassie blue detection of glaucomatous and normal optic nerve extracts and of antibody coupled beads as indicated. FIG. 4C, Western detection of anti-citrulline or anti-MBP IP products from glaucomatous and normal optic nerve extracts with anti-citrulline. FIG. 4D, Western detection of anti-citrulline or anti-MBP IP products from glaucomatous and normal optic nerve extracts with anti-MBP. FIG. 4E, Western detection of anti-citrulline IP products from glaucomatous optic nerve extracts with anti-myelin proteolipid protein (PLP), anti-myelin associated glycoprotein (MAG) and anti-MBP.

FIGS. 5A-5D. Elevated level of PAD2 and citrulline in response to pressure. FIG. 5A, Representative Western analyses with anti-PAD2 and anti-GPDH of human optic nerve demonstrating the presence of PAD2 relative to GPDH control immunoreactivity. Protein extracted from the optic nerve of cadaver Caucasian donor eyes, age and gender are as indicated. All glaucomatous donors suffered elevated IOP and were subjected to surgical intervention except the 76F donor. The 85M donor also received verapamil, a calcium modulator. FIG. 5B, Representative Western analyses with anti-PAD2 of rat brain astrocytes subjected to 40 mm Hg pressure for 5 h then returned to atmospheric pressure for up to 4 days as indicated. FIG. 5C, Representative Western analysis with anti-citrulline of protein extracts (5 μg) from astrocytes subjected to elevated pressure as in FIG. 5B. FIG. 5D, Representative Northern analyses of total RNA (˜2 μg) isolated from astrocytes pressure treated or untreated as in FIG. 5B.

FIGS. 6A-6C. Translational modulation of PAD2. FIG. 6A, Representative Northern analyses of total RNA (5 gg) isolated from normal control and glaucomatous human optic nerve. Donor age and gender are indicated. FIG. 6B, In vitro translation of PAD2 (measured as dpm) was monitored in polyA RNA, PAD2 and GPDH depleted normal control and glaucomatous optic nerve extracts. Radioactive PAD2 (relative to GPDH) is shown. FIG. 6C, Parallel Western analysis of in vitro translation products in FIG. 6B using anti-PAD2 and anti-GPDH with 700-IR coupled secondary antibodies. Grayscale images are from Odessey infrared scanning. Donor age and gender are indicated.

FIGS. 7A-7C. Transfection with shRNA restores PAD2 and citrullination to control levels in pressure treated astrocytes. Astrocytes were subjected to 40 mm Hg then transfected with PAD2 shRNA and analyzed for PAD2 expression and citrullination. The control is a non-silencing shRNA sequence. FIG. 7A, Anti-PAD2 Western analysis; FIG. 7B, Anti-citrulline Western analysis; FIG. 7C, Northern analysis of total RNA for PAD2 mRNA.

FIGS. 8A-8F. Immunohistochemical analysis of PAD 2 in isolated rat cortex astrocytes. Astrocytes were subjected to pressure (40 mm of Hg) for 5 hours and then to normal atmospheric pressure. Time of incubation in normal pressure is shown. Rat astrocyte controls not subjected to pressure are shown (FIG. 8A, 8D). Astrocytes were divided into two groups when subjected to normal pressure, untreated (FIG. 8B, 8C) or treated with shRNA for PAD2 (FIG. 8E, 8F) and stained with mouse monoclonal anti-PAD 2 and rabbit polyclonal GFAP.

FIGS. 9A-9B. Modulation of intracellular calcium concentration and PAD 2 expression. The astrocytes were subjected to 40 mm Hg for 5 hours and restored to normal pressure except controls. Pressure treated cells were subjected to (FIG. 9A) indicated concentrations of BAPTA-AM for 24 hours or (FIG. 9B) indicated concentrations of Thapsigargin. Total protein were extracted and transferred on PVDF membrane after SDS-PAGE separation and probed with antibodies to PAD 2 and GPDH, secondary antibodies coupled with IR-700 dye allowed scanning and detection.

FIG. 10. Western analysis for protein methyltransferases in optic nerve. Control and glaucomatous optic nerve protein (10 μg) were subjected to separation on SDS-PAGE and probed with monoclonal antibodies to PRMT1, CARM1 and GPDH. Protein extracted from the optic nerve of cadaver Caucasian donor eyes, age and gender as indicated.

FIG. 11A-11C. Immunohistochemical analysis of PAD2 in isolated rat cortex astrocytes. Astrocytes were subjected to pressure (40 mm of Hg) and stained with mouse monoclonal anti-PAD2 and rabbit polyclonal GFAP. FIG. 11A, Rat astrocyte controls (not subjected to pressure). FIG. 11B, Astrocytes 5 hours post pressure. FIG. 11C, Astrocytes 4 days post pressure treatment. Bar=40 μm

FIGS. 12A-12C. Immunocytochemistry using PAD2 and GFAP antibodies. FIG. 12A, Immunohistochemical analyses of astrocytes before and (FIGS. 12A-12C) 5 h and 4 days after pressure treatment. Post pressure treated cells were immediately treated with PAD2-shRNA. Bar=40 μm

FIG. 13 shows the deimination reaction in which PAD2 modifies arginine residues to citrulline, generating ammonia.

FIG. 14 is a diagram of shRNA against PAD2 (SEQ ID NO. 19).

FIG. 15 is the nucleotide sequence of human PAD2 (NM-007365) (SEQ ID NO.20).

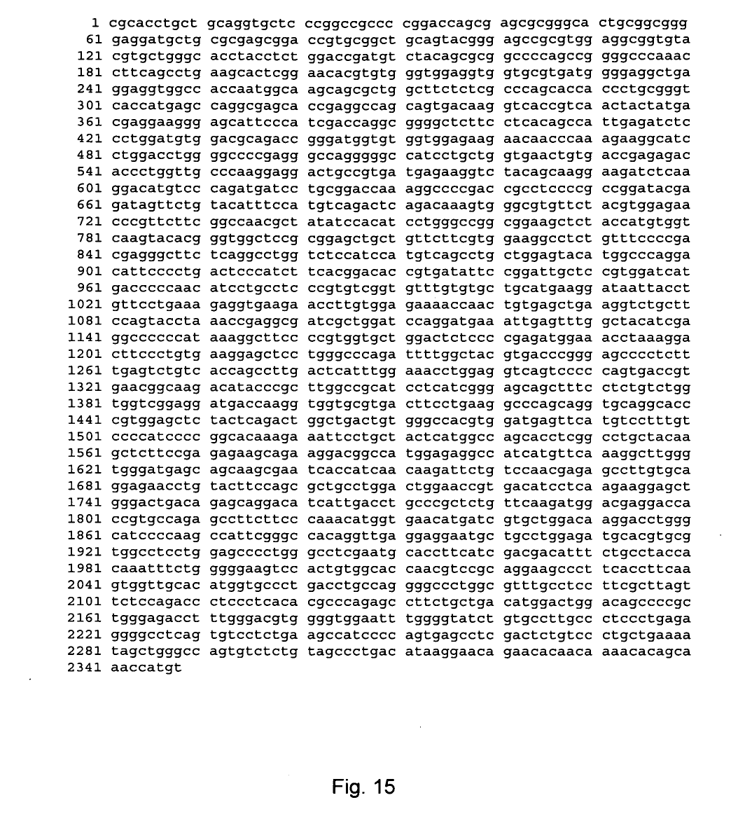

FIG. 16 is the amino acid sequence of human PAD2 (NM-007365) (SEQ ID NO.21).

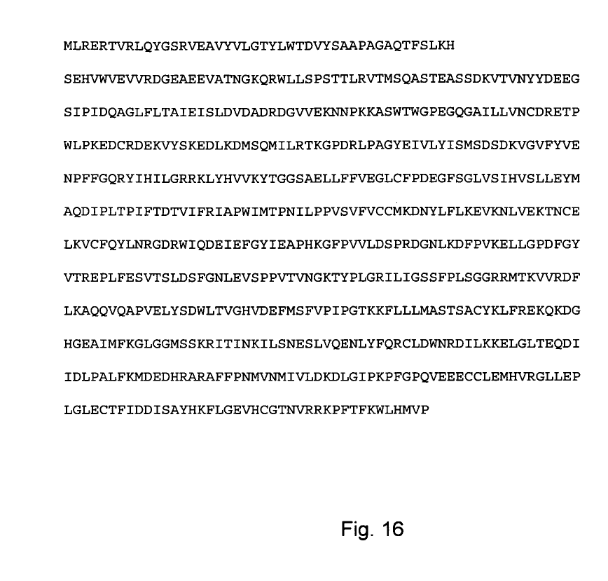

FIG. 17 is a bar graph showing inhibition of PAD2 activity by plant extracts.

### DETAILED DESCRIPTION OF THE INVENTION

As described herein, proteomic analyses of normal and glaucomatous human optic nerve were pursued for insights to the molecular pathology of primary open angle glaucoma (POAG). Peptidyl arginine deiminase 2 (PAD2), an enzyme that converts protein arginine to citrulline, was found only in POAG optic nerve and probed further for a mechanistic role in glaucoma. Protein identification utilized liquid chromatography tandem mass spectrometry. Northern, Western and immunohistochemical analyses measured PAD2 expression and/or protein citrullination and arginyl methylation in human and mouse optic nerve and in astrocyte cultures before and after pressure treatment. Proteins were identified following anti-citrulline immunoprecipitation. In vitro translation of PAD2 was monitored in polyA RNA depleted optic nerve extracts. PAD2 shRNA transfections were evaluated in pressure-treated astrocytes. Western and immunohistochemical analyses confirmed elevated PAD2 and citrullination in POAG optic nerve and decreased arginyl methylation. PAD2 was also detected in optic nerve from older, glaucomatous DBA/2J mouse, but not in younger DBA/2J or control C57BL6J mice. Myelin basic protein was identified as a major citrullinated protein in POAG optic nerve. Pressure treated astrocytes exhibited elevated PAD2 and citrullination without apparent change in PAD2 mRNA. Addition of exogenous polyA RNA to depleted optic nerve extracts yielded increased PAD2 expression in POAG but not in control extracts. Transfection with shRNA restored PAD2 and citrullination to control levels in pressure treated astrocytes. The results described herein show translational modulation of PAD2 expression and a role for the enzyme in POAG optic nerve damage through citrullination and structural disruption of myelination.

Primary open angle glaucoma (POAG) typically is associated with elevated intraocular pressure (IOP) and results in optic nerve damage also referred to as glaucomatous optic neuropathy (GON). Proteomic and Western analyses described herein demonstrate peptidyl arginine deiminase 2 (protein deiminase 2 or PAD2) in glaucomatous but not in normal optic nerve tissue. PAD2 converts arginine to citrulline. Glaucomatous optic nerve contains more citrullinated proteins and fewer methylarginine containing proteins than normal optic nerve. Others have associated PAD 2 with nerve damage in brain in experimental, drug induced animal models. PAD2 is known to be activated by calcium in the brain. PAD2 expression in POAG optic nerve is elevated as a consequence of elevated pressure. Once elevated, PAD2 expression is not reduced even by lowering pressure. Citrullination likely changes the structure and function of optic nerve proteins. PAD2 activity in the glaucomatous optic nerve contributes to the pathogenic mechanisms of POAG.

Thus, studies described herein indicate that increased PAD2 leads to and/or exacerbates optic nerve degeneration, and that without active intervention, increased PAD2 and consequent citrullination continue to exist in POAG optic nerve even when the pressure is reduced.

Based on these findings, provided herein is a method of inhibiting optic nerve damage in an individual in need thereof, comprising administering to the individual an agent that inhibits peptidyl arginine deiminase 2 (PAD2). In a particular embodiment, the present invention is directed to a method of inhibiting glaucomatous optic nerve damage in an individual in need thereof, comprising administering to the individual an agent that inhibits peptidyl arginine deiminase 2 (PAD2). The present invention is also directed to a method of treating glaucoma (e.g., primary open angle glaucoma) in an individual in need thereof, comprising administering to the individual an agent that inhibits (e.g., specifically inhibits) peptidyl arginine deiminase 2 (PAD2).

As used herein an “individual” includes mammals, as well as other animals, vertebrate and invertebrate (e.g., birds, fish, reptiles, insects (e.g., Drosophila species), mollusks (e.g., Aplysia). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and primates (e.g., monkeys, chimpanzees), canines (e.g., dogs), felines (e.g., cats), rodents (e.g., rats, mice, guinea pigs) and ruminents (e.g., cows, pigs, horses). For example, PAD2 is known to be expressed in mammals such as mouse (Q08642), rat (P20717), sheep (002849), chicken (BAA24913) and dog (XP_{—}544539).

The agent can inhibit expression of PAD2, biological activity of PAD2 or a combination thereof. Biological activity of PAD2 includes increased protein citrullination and decreased protein arginyl methylation. In a particular embodiment, the agent directly (specifically) inhibits the expression and/or biological activity of PAD2 (e.g., the agent is interfering RNA).

As used herein “optic nerve damage” refers to optic nerve damage associated with PAD2 expression and/or activity. In one embodiment, the optic nerve damage is associated with glaucoma and can be referred to as glaucomatous optic nerve damage. As indicated herein “glaucoma” refers to a group of late onset and progressive eye diseases that results in irreversible blindness often with no symptoms in the initial stages. Glaucomas are divided into two main categories: primary, where no apparent cause for onset can be attributed, and secondary, where an apparent cause such as previous injury or illness can be identified. Primary glaucoma is further divided into two groups, open angle (POAG), and angle-closure (PACG). POAG is the most common form of the disease, glaucoma affects about 3 million Americans and more than 70 million people worldwide (Thylefors, B., et al., Bull. World Health Organ., 73:115-121 (1995); Quigley, H. A., et al., Br. J. Ophthalmol., 80:389-393 (1996)). The risk of glaucoma has been found to increase with age, with glaucoma at age 80 being 5 to 10 times more prevalent than at age 40 (Wilson and Martone (1996) Epidemiology of Chronic Open-Angle Glaucoma. in Ritch R, Shields M B, Krupin T (eds), The Glaucomas. Mosby, St. Louis; Gordon, et al., 2002). Glaucoma are associated with optic neuropathy. In POAG most patients show elevated intraocular pressure (IOP) leading to optic nerve damage (Flammer, J. et al., Prog. Retin. Eye Res., 21:359-393 (2002)). Glaucoma is often equated with glaucomatous optic neuropathy (Van Buskirk, E. M., Invest. Ophthalmol. Vis. Sci., 22:625-632 (1982)). Many patients with glaucomatous optic neuropathy (GON) have increased IOP but not all patients with increased IOP suffer from GON (Flammer J. et al., Prog. Retin. Eye Res., 21:359-393 (2002)).

Elevated pressure on cultured cells from the optic nerve head (ONH) has been shown to modulate protein expression for example, nitric oxide synthase-2 (Neufeld, A. H., et al., Proc. Natl. Acad. Sci, USA, 96:9944-9948 (1999); Neufeld and Liu, Neurosci., 9:485-495 (2003)), elastin (Hernandez, M. R., et al., Glia, 32:122-136 (2000); Pena, J. D., et al., Invest. Ophthalmol. Vis. Sci., 42:2303-2314 (2001)), cytochrome P₄₅₀1 B I (Bejjani, B. A., et al., Exp. Eye Res., 75:249-257 (2002)), NCAM-180 (Ricard, C. S., et al., Brain Res. Mol Brain Res., 81:62-79 (2000)) and Hsp27 (Salvador-Silva, M., et al., J. Neurosci. Res., 66:59-73 (2001)). Expression of myocilin, a protein associated with glaucoma in optic nerve head is reduced in glaucoma as well as under conditions of elevated IOP (Ahmed, F., et al., Invest. Ophthalmol. Vis. Sci., 42:3165-3172 (2001); Clark, A. F., et al., Faseb J., 15:1251-1253 (2001); Ricard, C. S., et al., Exp. Eye Res., 73:433-447 (2001)). Described herein is a comparison of protein profiles between the optic nerve tissues from POAG and normal eyes which likely reflect the damage to the optic nerve in POAG. As shown herein, proteomic comparison has revealed a number of proteins associated with POAG (Bhattacharya, S. K., et al., ARVO Abstract, Ft. Lauderdale, Fla., p. 3510 (2005b)). Identification of in vitro differences in expressed proteins in response to pressure has recently been achieved by microarray analyses of the pressure treated and untreated astrocytes (Yang, P., et al., Physiol. Genomics, 17:157-169 (2004)). The proteomic analyses described herein have identified peptidyl arginine deiminase 2 (protein deiminase 2 or PAD 2) associated with POAG optic nerve. Previously, proteomic analyses of the aqueous outflow pathway associated cochlin in the trabecular meshwork with POAG (Bhattacharya, S. K., et al., Exp. Eye Res., 80:741-744,(2005a); Bhattacharya, S. K., et al., J. Biol. Chem., 280:6080-6084 (2005d)).

Optic nerve tissue environment is conducive to biochemical changes and protein modifications (Ingoglia, N. A., et al., J. Neurosci., 3:2463-2473 (1983); Chakraborty and Ingoglia, Brain Res. Bull., 30:439-445 (1993). Protein methylation and citrullination are among several posttranslational modifications (PTMs) found in the optic nerve that has important consequences in the function of multicellular organisms. They bring alteration in protein processing and signaling, protein-protein, protein-organelle, protein-cells and cell-cell interactions. The major function of cytosolic protein deiminases is citrullination (Vossenaar, E. R., et al., Bioessays, 25:1106-1118 (2003)). There are five known peptidyl arginine deiminases, all are cytosolic proteins (deiminase 1-3, 5 and 6), except PAD 4, which is nuclear (Nakashima, K. et al., J. Biol. Chem., 277:49562-49568 (2002); Cuthbert, G. L., et al., Cell, 118:545-553 (2004)). Recently PAD 4 was reported to reverse protein methylation by demethylimination (Cuthbert, G. L., et al., Cell, 118:545-553 (2004); Wang, Y., et al., Science, 306:279-283 (2004); Zhang, Y., Nature, 431:637-639(2004)). However, elevation in PAD 4 was not found in glaucomatous tissue in the analysis described herein. Citrullinated proteins, have been implicated in many diseases including autoimmune rheumatoid arthritis (Rubin and Sonderstrup, Scand. J. Immunol., 60:112-120 (2004); Scofield, R. H., Lancet, 363:1544-1546 (2004)), multiple sclerosis (MS) and amylotropic lateral sclerosis (ALS) (Chou, S. M., et al., J. Neurol. Sci., 139, Suppl. 16-26 (1996)). Citrullination has been also found in degenerating rat brain where PAD 2 activity has been implicated. In kainate induced neurodegeneration, citrullination remains confined only to degenerative regions of central nervous system (CNS) tissue (Asaga and Ishigami, Neurosci. Lett., 299:5-8 (2001); Asaga, H., et al., Neurosci. Lett., 326:129-132 (2002)).

At the optic nerve head (ONH) several proteins including matrix proteins are susceptible to citrullination. The optic nerve retrolaminar region is myelinated and amenable to protein modifications. Myelin is integral to the structure and function of optic nerve neurons at the retrolaminar region. The arginine residues of myelin basic protein (MBP), the major component of myelin (Carelli, V., et al., Neurochem. Int., 40:573-584 (2002)) undergoes citrullination. MBP has six arginine sites for this modification (Wood and Moscarello, J. Biol. Chem., 264:5121-5127(1989); Boggs, J. M., et al., Biochem., 36:5065-5071 (1997); Pritzker et al., (2000)). Citrullinated MBP and other proteins have been found in many neurodegenerative diseases such as MS and ALS (Moscarello, M. A., et al., J. Neurochem., 81:335-343 (2002)).

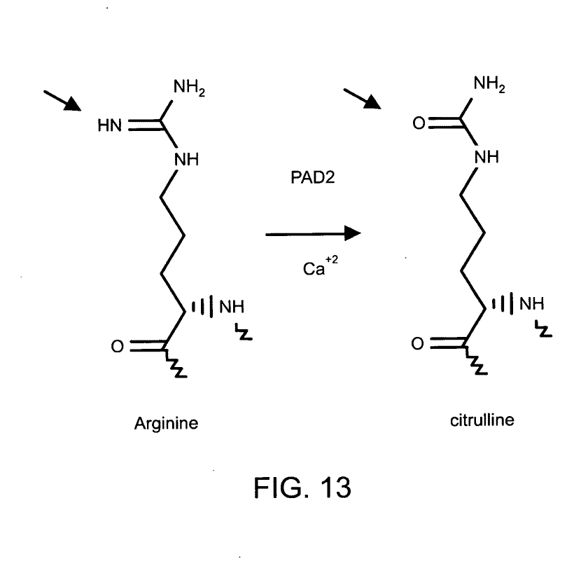

PAD 2 is predominantly expressed in neuronal tissues (Moscarello, M. A., et al., J. Neurochem., 81:335-343 (2002)). A variety of conditions including hypoxia (Sambandam, T., et al., Biochem, Biophys. Res. Commun., 325:1324-1329 (2004)) as well as pressure appears to trigger overexpression of PAD2 in astrocytes. In addition to astrocytes, observation of increased PAD 2 has been extended to myelinating immature oligodendrocytes (Akiyama, K., et al., Neurosci. Lett., 274:53-55 (1999)). As shown herein, PAD 2 modifies arginine residues to citrulline and generates ammonia in a process termed deimination (see FIG. 13).

However, arginine containing proteins have differences with respect to susceptibility to citrullination by PAD2 (Vossenaar, E. R., et al., Bioessays, 25:1106-1118 (2003)). At ONH annexins, lumican, mimecan, GFAP and decorin are among other proteins that appear to undergo citrullination in glaucomatous tissue. In the brain increased citrullination is implicated in demyelination and dysmyelination. Injuries to neurons may alter myelination and it has been shown possible to myelinate retinal ganglion cells upon injury that are normally non-myelinated (Setzu, A., et al., Glia, 45:307-311 (2004)). Myelination in the eye usually starts at the retrolaminar region but varies among donors. Injuries to neurons however, may render myelination at the level of the ONH as well (Setzu, A., et al., Glia, 45:307-311 (2004)). Initiation of glaucomatous neuropathy is believed to occur at the ONH. The implication of glaucomatous damage for myelination dynamics of the optic nerve remains poorly studied. Glaucomas are complex neuropathies and modification of myelin and other optic nerve proteins by several factors likely contributes to progression of neuropathy. It is also likely that citrullination of proteins at the ONH and progressive citrullination due to elevated PAD 2 level and subsequent subtle changes in the dynamics of myelin components have amplified consequences for vision. Citrullination likely brings changes in myelin dynamics that initiate progressive optic neuropathy. Alternatively, nerve damage is likely triggered by other factors but citrullination contributes to progression of glaucoma pathogenesis. Citrullination by PAD 2 is elevated by increased pressure and not reduced by lowering the pressure alone but requires active intervention. Elevated citrullination may be important in progressive optic nerve damage. The deiminase appears associated with cell cycle arrest events and apoptosis (Gong, h., et al., Biochem. Biophys. Res. Comm., 261:10-14 (1999); Gong, H., et al., Leukemia, 14:826-829 (2000)). Citrullination alters MBP (Boggs, J. M., Biochem., 36:5065-5071 (1997)). Other protein components of myelin also have been observed to undergo citrullination in different regions of the CNS.

PAD2 activity in damaged neuronal tissue is often triggered by calcium imbalance (Asaga and Ishigami, Neurosci. Lett., 299:5-8 (2001); Asaga, H., et al., Neurosci. Lett., 326:129-132 (2002)). Increased IOP in glaucoma is often associated with events (eg, ischemia) that induce excessive influx of calcium resulting in increased intracellular calcium (Osborne, N. N., et al., Surv. Ophthalmol., 43, Suppl. 1:S102-S108 (1999)). Hypoxia (and other sublethal injuries) also increases intracellular calcium concentration in astrocytes (Osborne, N. N., et al., Surv. Ophthalmol., 43, Suppl. 1:S102-S108 (1999)) and has been shown to increase PAD 2 level and citrullination in vitro (Sambandam, T., et al., Biochem. Biophys. Res. Comm., 325:1324-1329 (2004)). Calcium has been shown to modulate metabolism of astrocytes and oligodendrocytes. Intercellular calcium levels alter myelin gene expression (Studzinski, D. M., J. Neurosci., Res., 57:633-642 (1999)). Interaction of several myelin proteins (e.g., myelin-associated glycoprotein MOG) is modulated by calcium (Kursula, P., et al., J. Neurochem., 73:1724-1732 (1999); Marta, C. B., et al., J, Neurosci. Rers., 69:488-496 (2002)). Protein-protein interactions play key roles in the regulation of divalent cation-dependent signal transduction, myelin formation as well as maintenance of the myelin sheath. Interaction of the 18.5-kD isoform of MBP with calmodulin is modulated by citrullination of MBP (Libich, D. S., et al., Protein Sci., 12:1507-1521 (2003)). Events triggered by elevated IOP including increased intracellular calcium concentration likely increases the level of PAD2 in vivo and promote citrullination of optic nerve proteins.

Citrullination of the ONH matrix proteins may alter the ONH matrix. Altered and weak matrix may be susceptible for damage. Conversion of arginines to citrulline leads to loss of organized structures and protein-protein anchorage (Tarcsa, E., et al., J. Biol. Chem., 272:27893-27901 (1997)). The immunoprecipitation experiments described herein have revealed the presence of citrullinated annexins, mimecan, neurofilament H protein and GFAP in the optic nerve. The citrullination of matrix protein involved in anchorage leading to structural changes will weaken the optic nerve matrix. Consequences of citrullination include altered lipid vesicle formation by myelin components and apoptosis. Citrullinated MBP undergoes change in three dimensional structure and becomes more susceptible to digestion by cathepsin D (Pritzker, L. B., Biochem., 39:5382-5388 (2000)). The ability of modified MBP isomers to aggregate large unilamellar vesicles (LUVs) has been investigated. Citrullination decreases the ability of MBP to aggregate LUVs. Aggregation of acidic lipid vesicles by MBP is important for adhesion between intracellular surfaces of myelin. Thus charge modification by citrullination may affect adhesion in cytoplasm containing regions of myelin for example in the regions of paranodal loops where MBP concentration is low (Boggs, J. M., et al., Biochem., 36:5065-5071 (1997)). Increased susceptibility of citrullinated MBP to cathepsin D proteolysis may be one of the ways to generate immunodominant peptides leading to sensitization of T-cells for the autoimmune response in demyelinating diseases. Such mechanisms may play a role in glaucomatous neuropathy as well. Deiminase and citrullination also appear to inhibit proliferation leading to cell cycle arrest and apoptosis (Gong, H., et al., Biochem. Biophys. Res. Comm., 261:10-4 (1999); Gong, H., et al., Leukemia, 14:826-829 (2000)). Selective deimination of vimentin in calcium-ionophore induced apoptosis has been shown for mouse macrophages (Asaga, h., ET AL., Biochem. Biophys. Res. Comm., 243:641-646 (1998)). However, a more conclusive role for citrullination in events leading to apoptosis awaits more detailed investigation (van Venrooij and Pruijn, Arthritis Res., 2:249-251 (2000)). Nevertheless, the observation of citrullinated ONH matrix proteins and myelin proteins in glaucomatous tissue described herein indicates that PAD 2 and citrullination contribute to glaucoma pathogenesis.

### Methods of Therapy

Thus, the present invention pertains to methods of inhibiting optic nerve damage and methods of treatment (prophylactic, diagnostic, and/or therapeutic) for optic nerve damage (e.g., glaucomatous optic nerve damage) using a PAD2 therapeutic inhibitor compound or agent. In a particular embodiment, the invention is directed to methods of inhibiting glaucoma or treatment (prophylactic, diagnostic, and/or therapeutic) for glaucoma using a PAD2 therapeutic inhibitor compound or agent. A “PAD2 therapeutic inhibitor compound” is a compound that inhibits PAD2 polypeptide activity and/or PAD2 nucleic acid molecule expression, as described herein (e.g., a PAD2 antagonist). PAD2 therapeutic inhibitor compounds can alter PAD2 polypeptide activity or nucleic acid molecule expression by a variety of means, such as, for example, by altering post-translational processing of the PAD2 polypeptide; by altering transcription of PAD2; or by interfering with PAD2 polypeptide activity (e.g., by binding to a PAD2 polypeptide), or by downregulating the transcription or translation of the PAD2 nucleic acid molecule. Representative PAD2 therapeutic inhibitor compounds include the following: nucleic acids or fragments or derivatives and vectors comprising such nucleic acids (e.g., a nucleic acid molecule, cDNA, and/or RNA; polypeptides described herein; PAD2 substrates; peptidomimetics; fusion proteins or prodrugs thereof; antibodies (e.g., an antibody to PAD2); ribozymes; other small molecules; and other compounds that inhibit PAD2 nucleic acid expression or polypeptide activity, for example, those compounds identified in the screening methods described herein. One or more PAD2 therapeutic inhibitor compounds can be used concurrently (simultaneously) or sequentially in the methods of the present invention, if desired.

The terms, “inhibiting” and “treatment” as used herein, refer not only to ameliorating symptoms associated with the condition or disease, but also preventing or delaying the onset of the condition or disease, and also lessening the severity or frequency of symptoms of the condition or disease. The therapy is designed to inhibit (partially, completely) activity of PAD2 polypeptide in an individual. For example, a PAD2 therapeutic inhibitor compound can be administered in order to downregulate or decrease the expression or availability of the PAD2 nucleic acid molecule.

In a particular embodiment, the agent or compound that inhibits PAD2 activity is an antibody (e.g., a polyclonal antibody; a monoclonal antibody). For example, antibodies that bind all or a portion of PAD2 and that inhibit PAD2 activity (Koike, H. et al., Biosci. Biotechnol. Biochem., 58(12):2286-2287 (1994); Koike, H. et al., Biosci. Biotechnol. Biochem., 59(3):552-554 (1995)) can be used in the methods described herein. In a particular embodiment, the antibody is a purified antibody. The term “purified antibody” as used herein refers to immunoglobulin molecules and immunologically active portions of immunoglobulin molecules, i.e., molecules that contain an antigen binding site that selectively binds all or a portion (e.g., a biologically active portion of PAD2) of PAD2. A molecule that selectively binds to PAD2 is a molecule that binds to PAD2 or a fragment thereof, but does not substantially bind other molecules in a sample, e.g., a biological sample that naturally contains the PAD2 polypeptide. Preferably the antibody is at least 60%, by weight, free from proteins and naturally occurring organic molecules with which it naturally associated. More preferably, the antibody preparation is at least 75% or 90%, and most preferably, 99%, by weight, antibody. Examples of immunologically active portions of immunoglobulin molecules include F(ab) and F(ab′)2 fragments that can be generated by treating the antibody with an enzyme such as pepsin.

The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared using known techniques such as by immunizing a suitable subject with a desired immunogen, e.g., a PAD2 polypeptide or fragment thereof. The antibody titer in the immunized subject can be monitored over time by standard techniques, such as with an enzyme linked immunosorbent assay (ELISA) using immobilized polypeptide. If desired, the antibody molecules directed against the PAD2 polypeptide can be isolated from the mammal (e.g., from tissue, blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al., Immunol. Today 4:72 (1983)), the EBV-hybridoma technique (Cole et al., Monoclonal Antibodies and Cancer Therapy, Alan R. Liss, Inc., pp. 77-96 (1985)) or trioma techniques. The technology for producing hybridomas is well known (see generally Current Protocols in Immunology, Coligan et al., (eds.) John Wiley & Sons, Inc., New York, N.Y. (1994)). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature, 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

In one alternative to preparing monoclonal antibody-secreting hybridomas, a monoclonal antibody to a PAD2 polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9:1370-1372 (1991); Hay et al., Hum. Antibod. Hybridomas 3:81-85 (1992); Huse et al., Science 246:1275-1281 (1989); and Griffiths et al., EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

The antibodies of the present invention can also be used diagnostically to monitor PAD2 protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, and acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride and phycoerythrin; an example of a luminescent material includes luminol; examples of bioluminescent materials include luciferase, luciferin, green fluorescent protein, and aequorin, and examples of suitable radioactive material include, for example, 125I, ¹³¹, ³⁵S, ³²P and ³H.

In another embodiment, a nucleic acid of the invention can be used in the methods. For example, a nucleic acid of the invention can be used in “interfering RNA” therapy or in “antisense” therapy, in which a nucleic acid (e.g., an oligonucleotide) that specifically hybridizes to the RNA and/or genomic DNA of PAD2 is administered or generated in situ. The interfering RNA or antisense nucleic acid that specifically hybridizes to the RNA and/or DNA degrades and/or inhibits expression of the PAD2 nucleic acid molecule, e.g., by inhibiting translation and/or transcription.

An interfering RNA or antisense construct of the present invention can be delivered, for example, as an expression plasmid as described above. When the plasmid is transcribed in the cell, it produces RNA that is complementary to a portion of the mRNA and/or DNA that encodes a PAD2 polypeptide. Alternatively, the interfering RNA or antisense construct can be an oligonucleotide probe which is generated ex vivo and introduced into cells; it then inhibits expression by hybridizing with the mRNA and/or genomic DNA of PAD2. In one embodiment, the oligonucleotide probes are modified oligonucleotides that are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al., Biotechniques 6: 958-976 (1988); and Stein et al., Cancer Res 48: 2659-2668 (1988).

Endogenous PAD2 expression can also be reduced by inactivating or “knocking out” PAD2 nucleic acid sequences or their promoters using targeted homologous recombination (e.g., see Smithies et al., Nature 317: 230-234 (1985); Thomas and Capecchi, Cell 51: 503-512 (1987); Thompson et al., Cell 5: 313-321 (1989)). For example, a mutant, non-functional PAD2 (or a completely unrelated DNA sequence) flanked by DNA homologous to the endogenous PAD2 (either the coding regions or regulatory regions of PAD2) can be used, with or without a selectable marker and/or a negative selectable marker, to transfect cells that express PAD2 in vivo. Insertion of the DNA construct, via targeted homologous recombination, results in inactivation of PAD2. The recombinant DNA constructs can be directly administered or targeted to the required site in vivo using appropriate vectors, as described above.

Alternatively, endogenous PAD2 expression can be reduced by targeting deoxyribonucleotide sequences complementary to the regulatory region of PAD2 (i.e., the PAD2 promoter and/or enhancers) to form triple helical structures that prevent transcription of PAD2 in target cells in the body. (See generally, Helene Anticancer Drug Des., 6(6): 569-84 (1991); Helene et al., Ann, N.Y. Acad. Sci., 660: 27-36 (1992); and Maher, Bioassays 14(12): 807-15 (1992)).

The PAD2 therapeutic inhibitor compound(s) are administered in a therapeutically effective amount (i.e., an amount that is sufficient to treat the disease, such as by ameliorating symptoms associated with the disease, preventing or delaying the onset of the disease, and/or also lessening the severity or frequency of symptoms of the disease). The amount that will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

The therapeutic compounds can be delivered in a composition, as described above, or by themselves. They can be administered systemically, or can be targeted to a particular tissue. The therapeutic compounds can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein. A combination of any of the above methods of treatment can also be used.

The compounds for use in the methods described herein can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other compounds.

The composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active compound. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., that are, if desired, sterilized or mixed with auxiliary agents, e.g., preservatives, stabilizers, wetting agents, buffers or salts for influencing osmotic pressure, etc. The compound may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Compounds described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In another embodiment, the invention is directed to agents which inhibit PAD2 for use as a medicament in therapy. For example, the agents identified herein can be used in the treatment of optic nerve damage. In addition, the agents identified herein can be used in the manufacture of a medicament for the treatment of optic nerve damage.

### Screening Assays

The present invention also provides for a method of identifying an agent that can be used to inhibit optic nerve damage or treat glaucoma. The method comprises contacting a cell and/or animal which expresses peptidyl arginine deiminase 2 (PAD2) with an agent to be assessed. The level of expression or biological activity of PAD2 in the cell of animal is assessed, wherein if the level of expression or biological activity of PAD2 is decreased in the presence of the agent, then the agent can be used to inhibit intraocular pressure. In one embodiment, the biological activity of PAD2 that is assessed is citrullination and if citrullination is increased, then the agent can be used to inhibit optic nerve damage (e.g., optic nerve damage associate with glaucoma).

In the methods of the present invention the cell can be any suitable cell comprising nucleic acid which expresses PAD2. The cell can be a naturally occurring cell which comprises nucleic acid expressing PAD2 such as an ocular cell. For example, PAD2 is known to be expressed in mammals such as mouse (Q08642), rat (P20717), sheep (002849), chicken (BAA24913) and dog (XP_{—}544539). In a particular embodiment, the cell is an astrocyte. In another embodiment, the cell can be recombinantly produced. For example, exogenous nucleic acid which causes PAD2 to be expressed can be introduced into a cell that does not normally express PAD2.

Alternatively, an animal model can be used in the methods of the present invention. Any suitable animal which is a model for optic nerve damage can be used. For example, an animal model of glaucoma such as the DBA/2J mouse model can be used in the methods of the present invention.

The invention provides methods for identifying agents or compounds which include, for example, fusion proteins, polypeptides, peptidomimetics, prodrugs, receptors, binding agents, antibodies, small molecules or other drugs, or ribozymes that inhibit (e.g., partially (reduce, diminish), completely) the activity of PAD2. In a particular embodiment, the invention provides for identifying agents or compounds that inhibit optic nerve damage in an individual. For example, such compounds can be compounds or agents that bind to PAD2 described herein; that have an inhibitory effect on, for example, one or more activities of PAD2; or that inhibit the ability of PAD2 to interact with molecules with which PAD2 normally interact; or that alter post-translational processing of PAD2 polypeptide.

Methods for assessing the level of expression of PAD2 (e.g., SDS-PAGE, liquid chromatography/mass spectrometry (LC/MS)) or the biological activity of PAD2 (e.g., Western analysis) are provided herein and known in the art. Activities of PAD2 include increased protein citrullination, decreased protein arginyl methylation and combinations thereof. In the presence of the agent or compound identified herein, PAD2 activity can be decreased, for example, by at least 10%, at least 20%, 40%, 50%, 60%, 70%, 75%, 80%, 85%, 95%, 98%, or by at least 99%, relative to a control (e.g., PAD2 activity in the absence of the agent or compound).

As described herein PAD2 increases protein citrullination of a variety of optic nerve proteins listed in Table 2. In a particular embodiment, the optic nerve protein is a myelin protein. Examples of myelin proteins that can be citrullinated by PAD2 include myelin basic protein, myelin proteolipid protein, myelin associated glycoprotein, myelin P0 protein, myelin oligodendrocyte protein.

In one embodiment, the invention provides assays for screening candidate compounds or test agents to identify compounds that inhibit the activity of PAD2 (or biologically active portion(s) thereof), as well as agents identifiable by the assays. As used herein, a “compound”, “candidate compound”, “agent” or “test agent” is a chemical molecule, be it naturally-occurring or artificially-derived, and includes, for example, peptides, proteins, synthesized molecules, for example, synthetic organic molecules, naturally-occurring molecule, for example, naturally occurring organic molecules, nucleic acid molecules, and components thereof.

In general, candidate compounds for uses in the present invention may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. For example, candidate compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds (Lam, Anticancer Drug Des. 12: 145 (1997)). Furthermore, if desired, any library or compound is readily modified using standard chemical, physical, or biochemical methods. Compounds identified as being of therapeutic value may be subsequently analyzed using animal models for diseases in which it is desirable to alter the activity or expression of the PAD2 nucleic acids or polypeptides of the present invention.

In a particular embodiment, the methods can be used to determine whether an antibody which binds (specifically binds) to PAD2 is suitable for use in inhibiting optic nerve damage or treating glaucoma. Such antibodies can be obtained from commercial sources or produced using methods described herein and known in the art.

In another embodiment, the methods can be used to determine whether a nucleic acid such as a potential interfering RNA (e.g., siRNA, shRNA) is suitable for use in inhibiting optic nerve damage or treating glaucoma. Whether a particular interfering RNA down-regulates PAD 2 mRNA and/or delays optic nerve damage in DBA/2J mice can be accomplished using methods described in the exemplification and as known in the art. For example, shRNA (shRNA: hairpin RNA inhibitor generated from a vector; siRNA; inhibitor RNA oligonucleotide) is delivered to the optic nerve of DBA/2J mice either by expression in a construct (e.g., a lentiviral construct) or by direct injection (e.g., as siRNA); sham-treated mice eyes serve as controls. At specific time points, shRNA-treated and control mice are evaluated for IOP levels, the presence of optic nerve damage and the expression level of PAD 2 in the optic nerve. shRNA molecules that are effective for achieving this reduction are thereby identified.

In one embodiment, to identify candidate compounds that alter (e.g., inhibit) the biological activity of a PAD2 polypeptide, a cell, tissue, cell lysate, tissue lysate, or solution containing or expressing a PAD2 polypeptide or a biologically fragment of PAD2 or a derivative of PAD2, can be contacted with a candidate compound to be tested under conditions suitable for protein citrullination and/or arginyl methylation. Methods for assessing PAD2 activity are described herein. For example, methods of detecting citrullination and/or arginyl methylation are provided herein.

Alternatively, the PAD2 polypeptide can be contacted directly with the candidate compound to be tested. The level (amount) of PAD2 biological activity is assessed (e.g., the level (amount) of PAD2 biological activity is measured, either directly or indirectly), and is compared with the level of biological activity in a control (i.e., the level of activity of PAD2 polypeptide or active fragment or derivative thereof in the absence of the candidate compound to be tested, or in the presence of the candidate compound vehicle only). If the level of the biological activity in the presence of the candidate compound is reduced (lower), by an amount that is statistically significant, from the level of the biological activity in the absence of the candidate compound, or in the presence of the candidate compound vehicle only, then the candidate compound is a compound that inhibits the biological activity of a PAD2 polypeptide. In another embodiment, the level of biological activity of a PAD2 polypeptide or derivative or fragment thereof in the presence of the candidate compound to be tested, is compared with a control level that has previously been established. A level of the biological activity in the presence of the candidate compound that is lower than the control level by an amount that is statistically significant indicates that the compound inhibits PAD2 biological activity.

The present invention also relates to an assay for identifying compounds that inhibit the expression of a PAD2 nucleic acid molecule (e.g., interfering RNA (siRNA; shRNA), antisense nucleic acids, fusion proteins, polypeptides, peptidomimetics, prodrugs, receptors, binding agents, antibodies, small molecules or other drugs, or ribozymes) that decrease expression (e.g., transcription or translation) of the PAD2 nucleic acid molecule or that otherwise interact with the PAD2 nucleic acids, as well as compounds identifiable by the assays. For example, a solution containing a nucleic acid encoding a PAD2 polypeptide can be contacted with a candidate compound to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of PAD2 expression (e.g., the level and/or pattern of mRNA or of protein expressed) is assessed, and is compared with the level and/or pattern of expression in a control (i.e., the level and/or pattern of PAD2 expression in the absence of the candidate compound, or in the presence of the candidate compound vehicle only). If the level and/or pattern in the presence of the candidate compound is reduced, by an amount or in a manner that is statistically significant, from the level and/or pattern in the absence of the candidate compound, or in the presence of the candidate compound vehicle only, then the candidate compound is a compound that inibits the expression of PAD2. In another embodiment, the level and/or pattern of a PAD2 nucleic acids in the presence of the candidate compound to be tested, is compared with a control level and/or pattern that has previously been established. A level and/or pattern in the presence of the candidate compound that is reduced from the control level and/or pattern by an amount or in a manner that is statistically significant indicates that the candidate compound inhibits PAD2 expression.

In another embodiment of the invention, compounds that inhibit the expression of a PAD2 nucleic acid molecule or that otherwise interact with the nucleic acids described herein, can be identified using a cell, cell lysate, or solution containing a nucleic acid encoding the promoter region of the PAD2 gene operably linked to a reporter gene. After contact with a candidate compound to be tested, the level of expression of the reporter gene (e.g., the level of mRNA or of protein expressed) is assessed, and is compared with the level of expression in a control (i.e., the level of the expression of the reporter gene in the absence of the candidate compound, or in the presence of the candidate compound vehicle only). If the level in the presence of the candidate compound is reduced, by an amount or in a manner that is statistically significant, from the level in the absence of the candidate compound, or in the presence of the candidate compound vehicle only, then the candidate compound is a compound that inhibits the expression of PAD2, as indicated by its ability to alter expression of a gene that is operably linked to the PAD2 promoter. In another embodiment, the level of expression of the reporter in the presence of the candidate compound to be tested, is compared with a control level that has previously been established. A level in the presence of the candidate compound that is reduced from the control level by an amount or in a manner that is statistically significant indicates that the candidate compound inhibits PAD2 expression.

In one example, a cell or tissue that expresses or contains a compound that interacts with PAD2 (a PAD2 substrate such as a polypeptide or other molecule that interacts with PAD2) is contacted with PAD2 in the presence of a candidate compound, and the ability of the candidate compound to inhibit the interaction between PAD2 and the PAD2 substrate is determined, for example, by assaying activity of the polypeptide. Alternatively, a cell lysate, or a solution containing the PAD2 substrate, can be used. A compound that binds to PAD2 or the PAD2 substrate can alter the interaction by interfering with the ability of PAD2 to bind to, associate with, or otherwise interact with the PAD2 substrate. In a particular embodiment, the substrate is an optic nerve protein (e.g., myelin protein).

Determining the ability of the candidate compound to bind to PAD2 or a PAD2 substrate can be accomplished, for example, by coupling the candidate compound with a radioisotope or enzymatic label such that binding of the candidate compound to the polypeptide can be determined by detecting the label, for example, 125I, 35S, 14C, or 3H, either directly or indirectly, and the radioisotope detected by direct counting of radioemmission or by scintillation counting. Alternatively, candidate compound can be enzymatically labeled with, for example, horseradish peroxidase, alkaline phosphatase, or luciferase, and the enzymatic label detected by determination of conversion of an appropriate substrate to product.

In more than one embodiment of the above assay methods of the present invention, it may be desirable to immobilize a PAD2 nucleic acid, a PAD2 polypeptide, or a PAD2 substrate, or other components of the assay on a solid support, in order to facilitate separation of complexed from uncomplexed forms of one or both of the nucleic acids and/or polypeptides, as well as to accommodate automation of the assay. Binding of a candidate compound to the PAD2 nucleic acid or polypeptide, or interaction of the PAD2 nucleic acid or polypeptide with a substrate in the presence and absence of a candidate compound, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided that adds a domain that allows PAD2 or a PAD2 substrate to be bound to a matrix or other solid support.

In another embodiment, inhibitors of expression of nucleic acid molecules of the invention are identified in a method wherein a cell, cell lysate, tissue, tissue lysate, or solution containing a nucleic acid encoding PAD2 is contacted with a candidate compound and the expression of appropriate mRNA or polypeptide (e.g., variant(s)) in the cell, cell lysate, tissue, or tissue lysate, or solution, is determined. The level of expression of appropriate mRNA or polypeptide(s) in the presence of the candidate compound is compared to the level of expression of mRNA or polypeptide(s) in the absence of the candidate compound, or in the presence of the candidate compound vehicle only. The candidate compound can then be identified as an inhibitor of expression based on this comparison. The level of mRNA or polypeptide expression in the cells can be determined by methods described herein for detecting mRNA (e.g., Northern analysis) or polypeptide (e.g., Western analysis).

In another embodiment, the invention features a method of identifying a candidate compound that alters the expression level or biological activity of a PAD2 in an animal model. The method comprises contacting an animal with a candidate compound. The level of PAD2 mRNA or protein expressed or the biological activity of the protein is assessed, and is compared with the level of expression or biological activity in a control (e.g., the level of the expression or biological activity in the absence of the candidate compound, or in the presence of the candidate compound vehicle only) using, for example, methods described herein. If the level of expression or activity in the presence of the candidate compound is reduced, by an amount or in a manner that is statistically significant, from the level in the absence of the candidate compound, or in the presence of the candidate compound vehicle only, then the candidate compound is a compound that inhibits the expression or biological activity of PAD2. In one embodiment, the biological activity is assessed by detecting a decrease in protein citrullination of an optic protein.

This invention further pertains to novel compounds identified by the above-described screening assays. A compound identified as described herein (e.g., a candidate compound that is an inhibiting compound such as an antisense nucleic acid molecule, a specific antibody, or a polypeptide substrate) can be used in an animal model to determine the efficacy, toxicity, or side effects of treatment with such a compound. Alternatively, a compound identified as described herein can be used in an animal model to determine the mechanism of action of such a compound. Furthermore, this invention pertains to uses of novel compounds identified by the above-described screening assays for treatments as described herein.

The present invention is also directed to a method of detecting optic nerve damage) in an individual comprising detecting the presence of peptidyl arginine deiminase 2 (PAD2) in the individual's optic nerve, wherein if the presence of PAD2 in the individual's optic nerve is higher than the presence of PAD2 in a control, then glaucoma is detected in the individual. In a particular embodiment, the present invention is directed to a method of detecting glaucoma (e.g., primary open angle glaucoma) in an individual comprising detecting the presence of PAD2 in the individual's optic nerve. The PAD2 can be detected in a lamina cibrosa region of the optic nerve. The PAD2 detected can be an increased amount of PAD2 compared to a suitable control.

The presence of PAD2 is detected by measuring PAD2 expression, protein citrullination, protein arginyl methylation or a combination thereof.

The present invention is also directed to a method of determining whether an individual is at risk for developing glaucoma comprising detecting the presence of peptidyl arginine deiminase 2 (PAD2) in the individual's optic nerve, wherein if the presence of PAD2 in the individual's optic nerve is higher than the presence of PAD2 in a control, then the individual is at risk for developing glaucoma.

Also encompassed by the present invention is a method of monitoring a treatment regimen for glaucoma comprising detecting the presence of peptidyl arginine deiminase 2 (PAD2) in the individual's optic nerve, wherein if the presence of PAD2 in the individual's optic nerve is lower after treatment, then the treatment regimen is successful.

The invention will be further described by the following non-limiting examples. The teachings of all publications cited herein are incorporated herein by reference in their entirety.

### Example 1

### PAD2 and Optic Nerve Citrullination in Glaucoma Pathogenesis

### Methods

### Tissue Procurement

Donor eyes from normal (control) and POAG cadavers were enucleated within 6 h of death and obtained from the National Disease Research Interchange and the Cleveland Eye Bank. Glaucomatous eyes that had recorded optic neuropathy and progressive deterioration in visual acuity together with lack of other major CNS disorder were procured. Acceptable eyes were those that had detailed medical and ophthalmic histories. Control eyes were from normal donors that lacked optic neuropathy and had no history of eye diseases or other major CNS disorder. Twelve glaucomatous and 12 age-matched (±4 years) normal eyes, all from Caucasian donors between 55-87 years of age were used in this study. Two additional eyes from different 7 year old Caucasian male donors were used for astrocyte cell culture preparation. Research was conducted following the tenets of the Declaration of Helsinki. Use of mice followed procedures in adherence to the ARVO statement for the use of animals in ophthalmic and vision research.

### Protein Identification, Western Analysis, Immunohistochemical Analysis, Immunoprecipitation and Protein Methylation Assays

Briefly, for proteomic analyses, proteins were extracted from optic nerve as reported previously with minor modifications (Bhattacharya, S. K., et al., Separation Methods in Proteomics (2005)) and proteins identified by liquid chromatography tandem mass spectrometry and bioinformatics methods (Bhattacharya, S. K., et al., J. Biol. Chem., 280:6080-6084 (2005)). Western analysis utilized PVDF membrane, established protocols (Bhattacharya, S. K., et al., J. Biol. Chem., 280:6080-6084 (2005); Bhattacharya, S. K., et al., Separation Methods in Proteomics (2005)) and primary antibodies to PAD2, myelin basic protein (MBP), myelin proteolipid protein (PLP), myelin associated glycoprotein (MAG), gligal fibrillary acidic protein (GFAP), citrulline and methyl arginine. Immunohistochemical analyses to localize PAD2 and citrulline in optic nerve tissue utilized cadaver eyes enucleated within six hours of death and fixed immediately with calcium acetate buffered 4% para-formaldehyde. Immunoprecipitations were performed using antibodies to citrulline and myelin basic protein covalently coupled to protein A sepharose beads with dimethylpimelimidate. Protein methylation assays were performed by measuring incorporation of S adenosyl-L-methyl-¹⁴C methionine into ovalbumin using standard protocols.

### Proteomic Analyses

Briefly, optic nerve tissues were minced with an angled scissor and extracted by homogenization in 100 mM Tris-Cl buffer pH 7.8 containing 5 mM dithiotheritol, 1 mM SnCl₂, 50 mM NaHPO₄, 1 mM diethylenetriaminepentaacetic acid, 100 mM butylated hydroxyl toluene and 0.2% SDS. SDS was replaced by 0.1% genapol for extracts where enzymatic determinations were required. Insoluble material was removed by centrifugation (8000×g for 5 min), and soluble protein quantified by the Bradford assay (Bradford, M. M., Anal Biochem. 72:248-54(1976)). Protein extracts were subjected to SDS-PAGE on 10% gels (Bio-Rad Laboratories, Hercules, Calif.) and the gels were used either for mass spectrometric proteomic analyses or for Western analyses. For protein identifications, gel slices were excised and digested in situ with trypsin, and peptides were analyzed by liquid chromatography electrospray tandem mass spectrometry using a CapLC system and a quadrupole time-of-flight mass spectrometer (QTOF2, Waters Corporation, Milford, Mass.). Protein identifications from MS/MS data utilized ProteinLynx™ Global Server (Waters Corporation) and Mascot (Matrix Science) search engines and the Swiss-Protein and NCBI protein sequence databases (Bhattacharya, S. K., et al., J Biol Chem.; 280:6080-6084. (2005).

### Western Analyses

For these analyses previously described mouse monoclonal antibody (mAb) against PAD2 (Koike, H., et al., Biosci Biotechnol Biochem. 58:2286-7(1994); Koike, H., et al., Biosci Biotechnol Biochem. 59:552-4(1995)) was used. Mouse mAbs for human myelin basic protein (MBP), myelin proteolipid protein (PLP), myelin associated glycoproteins (MAG) and glial fibrillary acidic protein (GFAP) were procured from Chemicon International unless stated otherwise. For quantitative Western analyses, anti-mouse and anti-rabbit secondary antibody linked to 700 nm or 800 nm IR-dyes were used on an Odyssey Infrared Imaging system according to the manufacturer (Li-Cor Biosciences, Lincoln, Neb.). Polyclonal antibodies (pAbs) to citrulline (Citrulline kit, Upstate Biotechnology), and methyl arginine antibodies (ab412, Abcam) were purchased.

### Protein Methylation Assays

Protein methylation assays were performed by measuring incorporation of S adenosyl-Lmethyl-¹⁴C methionine (AdoMet; Sigma Chemical Co. St. Louis, Mo.). AdoMet (¹⁴-Clabeled; specific activity 50 m Ci/mM) was diluted to yield a concentration of 0.1 mM (100-150 dpm/picomole) and allowed incorporation into the proteins (Ovalbumin) at pH 7.2 following standard protocols (Hyun, Y. L., et al., Biochem J. 348 Pt 3:573-8 (2000)). AdoMet was incubated with Ovalbumin at 37° C. for 5 minutes and the reaction was initiated by adding 5 μl of protein extract (1 mg/ml) and incubated for an additional 5 minutes. The reaction was stopped by adding 0.5 ml of 30% TCA. In control tubes, an equivalent amount of ovalbumin instead of tissue extract was added. The mixture was carefully overlayed with ethanol and centrifuged for 15 minutes in a tabletop clinical centrifuge. The supernatant was decanted and the precipitate was washed three times with 8 ml of TCA solution, once with chloroform:ether:ethanol (1:1:1 v/v), and once with ethanol. The precipitates were dissolved in 1 ml of 0.2 M sodium phosphate buffer (pH 7.2) by placing it in a boiling water bath for 5 minutes then transferred into 10 ml of scintillation fluid and counted for radioactivity. One ml of 0.2 M sodium phosphate buffer (pH 7.2) in a tube served as a blank control. The protein methylase activity was determined for three samples each of equal amounts (10 μg) of tissue extract from control and glaucomatous optic nerve.

### Immunoprecipitations

Antibody-coupled protein A beads were used for all immunoprecipitations (IPs). About 100 μg of protein A sepharose CL-4B beads (Amersham Pharmacia Biotech, CA) was coupled with 100 μg antibody (citrulline or MBP) using dimethylpimelimidate (DMP). The antibody-bead suspension was subjected to addition of 25 mg of DMP and incubated at room temperature in 50 mM sodium borate buffer pH 8.3 for 2 hour, the addition of 25 mg DMP to the suspension was repeated 4 times. Rabbit pAb against human MBP, procured from Dako Corporation was used for IP and mouse human MBP mAb was used for Western detection. Antibody-conjugated beads were washed and incubated for 2 hour with 200 mM ethanolamine pH 8.0. Antibody beads were finally washed with phosphate buffered saline pH 7.4 and incubated with protein extracts (˜100 μg) prepared in 100 mM Tris-Cl buffer pH 7.5, 50 mM NaCl and 0.01% genapol. For IP with anti-citrulline, the protein extract in a total volume of 10 μl (2-2.5 μg/μl) was treated with 2 μl of acidified FeCl₃ containing 2,3-butanedione monooxime and antipyrine provided in the citrulline kit for 90 minutes. Time period of 90 minutes was found optimal and prevents formation of insoluble materials. Following incubation the volume was raised to 500 μl using 100 mM Tris-Cl buffer pH 8.0, 50 mM NaCl and 0.01% genapol and incubated with 100 μg of anti-citrulline coupled beads for 1 hour at room temperature. The MBP IP was performed by incubating 100 μg antibody-coupled beads with ˜100 μg protein extract in 500 μl of 100 mM Tris-Cl buffer pH 7.5, 50 mM NaCl and 0.01% genapol for 1 hour. After incubation the beads were recovered by centrifugation at 2500×g for 5 minutes and washed with 3×500 μl of 100 mM Tris-Cl buffer pH 7.5, 100 mM NaCl and 0.02% genapol. The beads were boiled with 30 μl Laemmli buffer (Laemmli, U.K., Nature 227:680-5 (1970)) for 2 minutes and separated on a 10% SDS-PAGE. The gels were subjected to either Western blot analyses or Coomassie blue staining with subsequent LC MS/MS of excised gel bands.

### Histochemical Analyses

Immunohistochemical analyses to localize PAD2 in optic nerve tissue were performed with cadaver eyes enucleated within six hours of death and fixed immediately with calcium acetate buffered 4% para-formaldehyde. Paraffin embedded tissue was blocked and sectioned (12 μm) in 2% BSA in phosphate buffered saline (PBS), then incubated with 10 ng anti-PAD2 antibody (Koike, H., Shiraiwa, et al., Biosci Biotechnol Biochem. 59:552-4 (1995)) overnight at 4° C. and subsequently with 10 ng Alexa 594 conjugated secondary antibody (Jackson ImmunoResearch Laboratories Inc., West Grove, Pa.) for one hour at room temperature. For immunohistochemical analysis of citrulline containing proteins, a kit from Upstate Biotechnology was used. Briefly, the tissue sections after de-paraffinization were subjected to 2,3-butanedione monooxime and antipyrine treatment in a strong acid atmosphere for 3 hours followed by five washes with 2% BSA in PBS. For detection of citrulline, Alexa 488 conjugated secondary antibody was used. The nuclei were stained with TOPRO-3. The treatment of tissue with 2,3butanedione monooxime and antipyrine in a strong acid atmosphere enables chemical modification of citrulline into ureido groups and ensures detection of citrulline-containing proteins regardless of neighboring amino acid sequences (Senshu, T., Sato, et al., Anal Biochem. 203:94-100 (1992)). Processing steps, in a strong acid environment and antipyrine, however, makes TOPRO-3 nuclear staining (or any other nuclear stain) less pronounced. Sections sealed with vectashield and were analyzed either with a Leica TCP2 scanning confocal microscopeor with a Nikon EFD-3 fluorescence microscope attached to a CCD camera. Rat brain astrocytes were subjected to immunohistochemical analysis in a similar fashion.

### Western Analysis of PAD2 and Citrulline in the Mouse Optic Nerve

DBA/2J Mice were procured from The Jackson Laboratory (Bar Harbor, Me.) and bred to generate the animals used in this study. Mice were sacrificed with carbon dioxide and optic nerve tissue was dissected. All procedures were approved by the Institutional Animal Care and Use Committee of the Cleveland Clinic Foundation. Protein was extracted from optic nerve tissue by homogenization in 100 mM Tris-Cl buffer pH 7.5 containing 5 mM dithiotheritol, 1 mM SnCh, 50 mM NaHPO₄, 1 mM diethylenetriaminepentaacetic acid, 100 mM butylated hydroxy toluene and 0.5% SDS. Insoluble material was removed by centrifugation (8000×g for 5 min), and soluble protein quantified by the Bradford assay. Western blot analyses were performed with 5 μg protein extract, 4-20% gradient gels (Invitrogen Inc, CA), electroblotting to PVDF membrane and probing with monoclonal PAD2 antibody or polyclonal anti-citrulline antibody.

### RNA Isolation and Quantitation

RNA isolation was performed using TRIZOL with suitable modification of standard protocols. Northern analyses were probed with ³²P-CTP labeled PCR products and after 1 hour exposure to a Molecular Dynamics Phosphorimager screen, imaged using a Typhoon 8600 variable mode imager with Imagequant software. Probes for PAD2 (5′-aaacctggaggtcagtcccc-3′ (SEQ ID NO: 1) and 5′-aaacctggaggtcagtcccc-3′ (SEQ ID NO: 2)), GPDH (5′-cttcaccaccatggagaaggc-3′ (SEQ ID NO: 3) and 5′-ggcatggactgtggtcatgag-3′ (SEQ ID NO: 4) and HGRT (5′-gaagagctactgtaatgatcagtc-3′ (SEQ ID NO: 5) and 5′-aaagtctggcctgtatccaacac-3′ (SEQ ID NO: 6)) were generated by PCR for 30 cycles using the indicated primer pairs, 32P-CTP (9.25 MBq/25 μl) and recommended protocols (Sambrook, J., et al., Molecular Cloning: A Laboratory Manual, NY:Cold Spring Harbor Laboratory Press (1989)).

### RNA Isolation and Quantitation

Total RNA from optic nerve was isolated using TRIZOL with modification of the protocol recommended by the supplier (Invitrogen Inc., Carlsbad, Calif.). The optic nerve from donor eyes was carefully excised and minced into small pieces first using scissors and then a scalpel. Prior to use, tissue was washed with diethylpyrocarbonate (DEPC) water and all solutions were prepared in DEPC water. The minced tissue was placed in a glass homogenizer with 1 ml TRIZOL per 100 mg of tissue and homogenized in a glass homogenizer DUALL 20 (Kimble Kontes Glass Co, Vineland, N.J.) with 10 stroke cycles each at room temperature and after freezing with liquid nitrogen for 1 min for 40 cycles. This RNA was extracted with chloroform, isoamylalcohol and precipitated with sodium citrate/sodium chloride and isopropanol. The RNA from astrocytes was isolated following the standard recommended TRIZOL protocol. The final air-dried RNA precipitate was suspended in DEPC water, spectrophotometrically quantified and stored at −80° C. until use. For relative quantification, about 1 μg of RNA after separation on a 5% polyacrylamide gel in TAE buffer was subjected to Northern blotting using standard protocols (Sambrook, J., et al., Molecular Cloning: A Laboratory Manual. New York: Cold Spring Harbour Laboratory Press, (1989)). It was probed with ³²P-CTP labeled PCR products and after 1 hour exposure to a Molecular Dynamics Phosphorimager screen, imaged using a Typhoon 8600 variable mode imager with Imagequant software. Probes for PAD2 (5′-aaacctggaggtcagtcccc-3′ and 5′-aaacctggaggtcagtcccc-3′), GPDH (5′-cttcaccaccatggagaaggc-3′ and 5′ggcatggactgtggtcatgag-3′) and HGRT (5′-gaagagctactgtaatgatcagtc-3′ and 5′ aaagtctggcctgtatccaacac-3′) were generated by PCR for 30 cycles using the indicated primer pairs, ³²P-CTP (9.25 MBq/25 μl) and recommended protocols (Sambrook, J., et al., Molecular Cloning: A Laboratory Manual. New York: Cold Spring Harbour Laboratory Press, (1989)).

### Primary Astrocyte Cultures and Pressure Treatment

Astrocytes from Sprague Dawley rat (Harlan, Indianapolis, Ind.) brain cortex were used for these studies. Mixed glial cell suspensions were prepared from the third postnatal day (P3) rat brain cortex region following published procedures (Fuss, B., et al., Dev. Biol., 218:259-274 (2000)) from which enriched GFAP positive cells were obtained by immunopanning (Yang, et al., Brain Res. Brain Res. Protocol, 12:67-76 (2003)).

### Primary Astrocyte Cultures and Pressure Treatment

Astrocytes from Sprague Dawley rats (Harlan, Indianapolis, Ind.) brain cortex were used for these studies. Mixed glial cell suspensions were prepared from the P3 rat cortex regions following published procedures (Fuss, B., et al., Dev Biol., 218:259-74 (2000)) from which enriched GFAP positive cells were obtained using immunopanning (Yang, P., et al., Brain Res Brain Res Protoc. 12:67-76 (2003)). The astrocytes were exposed to a pressure of 40 mm of Hg for five hours (Yang, J. L., et al., Exp Eye Res. 56:567-74 (1993); Salvador-Silva, M., et al., Glia. 45:364-77 (2004)). Briefly, the cells plated in six well plates (Costar, Cambridge, Mass., USA) at a density of 3-10×10³ cells/well and grown to semiconfluence in 2 days were incubated with serum free medium overnight. A closed pressurized chamber (5% carbon dioxide) equipped with a manometer was used to subject the cells to elevated pressure. Cells were placed in the chamber and the pressure was elevated to 40 mm Hg. The chamber was subsequently placed in a tissue culture incubator at 37° C. Control cells from identical passage of cell lines were simultaneously incubated in a tissue culture incubator at atmospheric pressure at 37° C. The cells were incubated for 5 h or 1-4 days after pressure treatment. After incubation, cells were trypsinized and subjected to culture or Western analyses. For culture, the cells were plated on a cover slip and allowed 16 hours recovery period and subjected to immunohistochemistry using mouse monoclonal PAD2 and rabbit polyclonal GFAP antibodies. The cells were permeabilized with 200 μl of 0.2% Triton X-100 in phosphate buffer saline pH 7.5 for 1 hour after fixation with 4% paraformaldehyde for 1 hour. Western analyses were performed using antibodies to PAD2 and citrulline as described above.

Probing Translation with Poly Adenylated RNA Depleted Extracts

Assays were performed probing translation of PAD2 upon addition of total polyA RNA to optic nerve extracts depleted of mRNAs, PAD2 and GAPDH. Extracts were first depleted of poly A RNA with oligo dT-cellulose matrix (BioWorld, Dublin, Ohio) then depleted of PAD2 and GPDH using mAb and pAb to PAD2 and GPDH, respectively, conjugated to protein A Sepharose beads. About 100 μg total protein so obtained from each donor was used for each analysis. About 0.15 μg total poly A RNA (pooled from two nerves from Caucasian males, 79 years and 70 years) was added to poly A RNA depleted tissue extracts (per 100 μg of extract) and incubated at 37° C. for 90-120 minutes. ³⁵S labeled methionine (540 Ci/mmol; MP Biomedical Inc, CA) was used to detect the translated product. Two identical gels (SDS-PAGE), one with ³⁵S labeled and the other with cold methionine was subjected to simultaneous side-by-side electrophoresis. The cold methionine gel was blotted to PVDF membrane (Sigma Chemical Co., St. Louis, Mo.) and probed with PAD2 and GPDH antibodies to determine the identity of protein bands. Detection utilized IR-700 or IR-800 dye coupled secondary antibodies (Vosseenaar, E. R., et al., Bioessays, 25:1106-1118 (2003)). The radioactive protein bands corresponding to antibody detected counterparts in cold methionine gels were excised and quantified in a scintillation counter (TRI-CARB, 19000A).

The shRNA Treatment of Primary Astrocytes

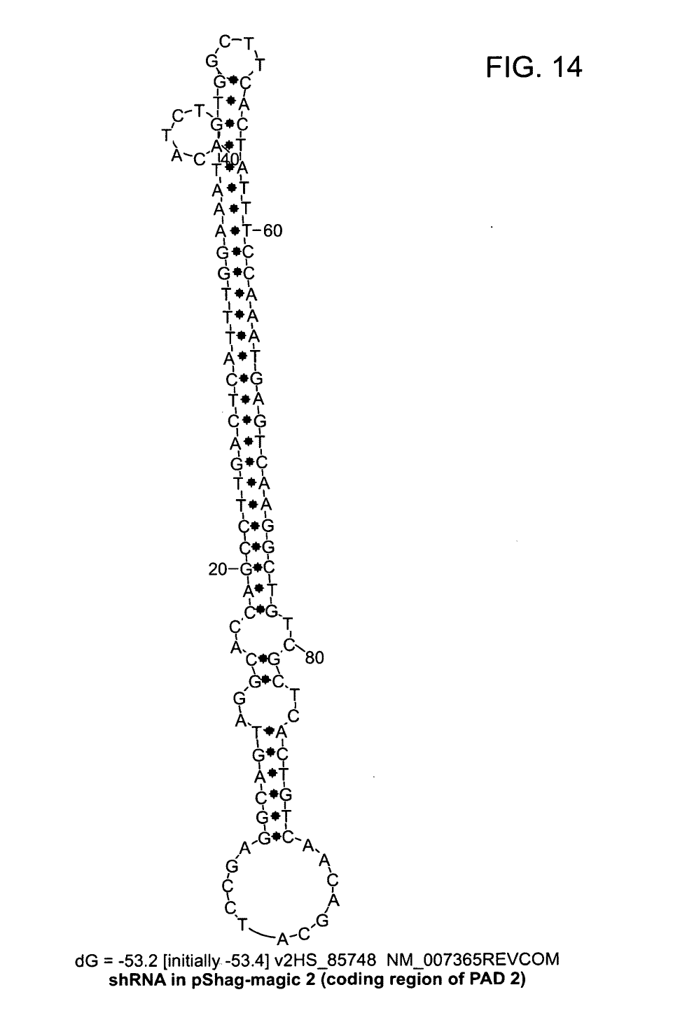

For treatment of rat cortex astrocytes, shRNA against PAD2 (OpenBiosystems, Cat. #RHS 1764-9214220) was procured from OpenBiosystems in pShag Magic version 2.0 vector (OpenBiosystems). This shRNA contains the sequence (5′TGCTGTTGACAGTGAGCGACAGCCTTGACTCATTTGGAAATAGTGAAGCCA CAGATGTATTTCCAAATGAGTCAAGGCTGGTGCCTACTGCCTCGGA′3) (SEQ ID NO: 7) from the coding region of PAD2 (see FIG. 14). For a negative control, we used a non-silencing shRNA sequence cloned into pShag Magic 2.0 (OpenBiosystems, cat. #RHS1703) from OpenBiosystems verified to contain no homology to known mammalian genes. About 30% confluent cells (3000 primary astrocytes) (Lee, J. H., et al., Glia., 50:66-79 (2005)) were transfected using SuperFect transfection reagent, purified vector (5 μg) DNA (Qiagen, Valencia, Calif.) and the manufacturer's recommended protocols. The post transfected astrocytes were selected on geneticin (10 μg/μl). The primary astrocytes were subjected to pressure (40 mm of Hg) and transfected with shRNA on plates immediately after they were brought to normal atmospheric pressure.

### Results

### Detection of Peptidyl Arginine Deiminase 2 in Glaucomatous Optic Nerve

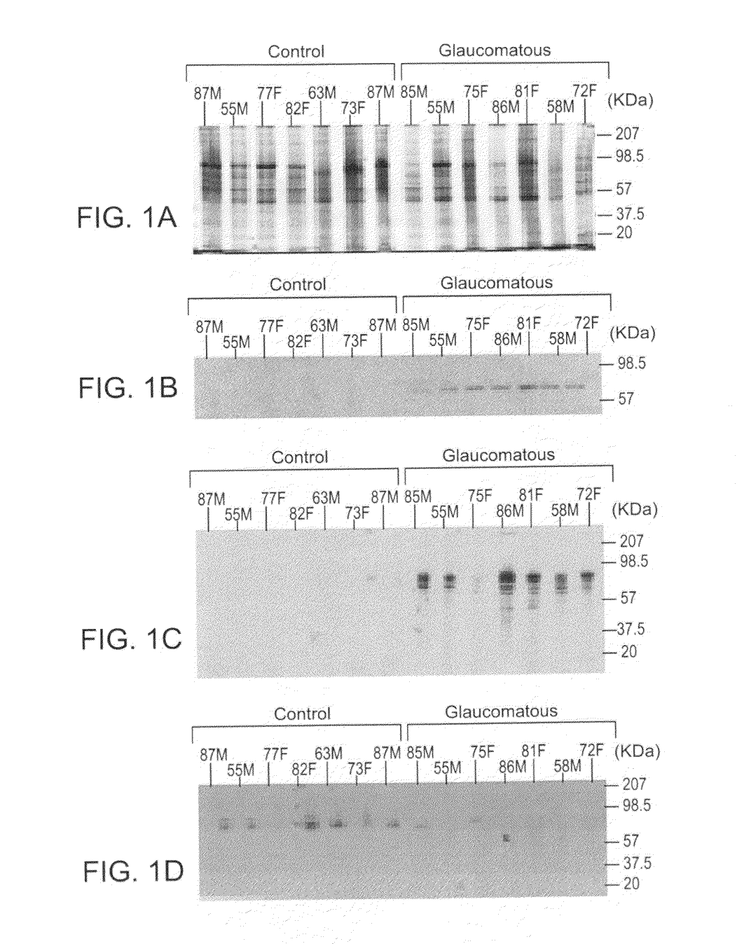

Protein extracts from eight POAG and eight control optic nerve donor tissues were separated on SDS-PAGE, gel slices were excised from the top to the bottom of the gel (FIG. 1A) and proteins were identified using well-established mass spectrometric and bioinformatics methods. Two additional donor tissues not shown in FIG. 1A (Caucasian females, POAG and control, age 72 and 73 years respectively) were also subjected to proteomic analyses. Overall, 250 proteins were identified, of which 68 were detected only in glaucomatous optic nerve (Table 1). Apparent proteome differences must be verified because the lack of detection by LC MS/MS does not necessarily mean absence of protein expression. Notably, PAD2 was detected in 4 of 8 glaucomatous optic nerve by mass spectrometry (Table 1) and subsequently by immunoblotting verified in 7 of 7 glaucomatous but not detected in any normal optic nerve tissue (FIG. 1B). Western analyses of five additional glaucomatous tissues also detected PAD2. Overall, it was found that PAD2 uniquely associated with 12 of 12 POAG donor optic nerves by proteomic and Western analyses combined but in none of 12 normal controls devoid of other neurodegenerative disorders. However, by immunoblot we did detect PAD2 in optic nerve from 2 of 7 human donors exhibiting other CNS disorders but without glaucoma, a finding consistent with reports of increased PAD2 in several neurodegenerative diseases (Asaga, H., et al., Neurosci. Lett., 326:129-132 (2002); Asaga, H., et al., Neurosci. Lett., 299:5-8 (2001)). See Table 3 for a listing of the optic nerve tissue donors.

Based on these findings from human optic nerve, PAD2 expression was probed in an established animal model of glaucoma, the DBA/2J mouse. This mouse line exhibits increased IOP around 6-8 months of age, with progressive damage to the optic nerve and hearing loss (John, S. W., et al., Invest. Ophthalmol. Sci., 38:249-253 (1997)).

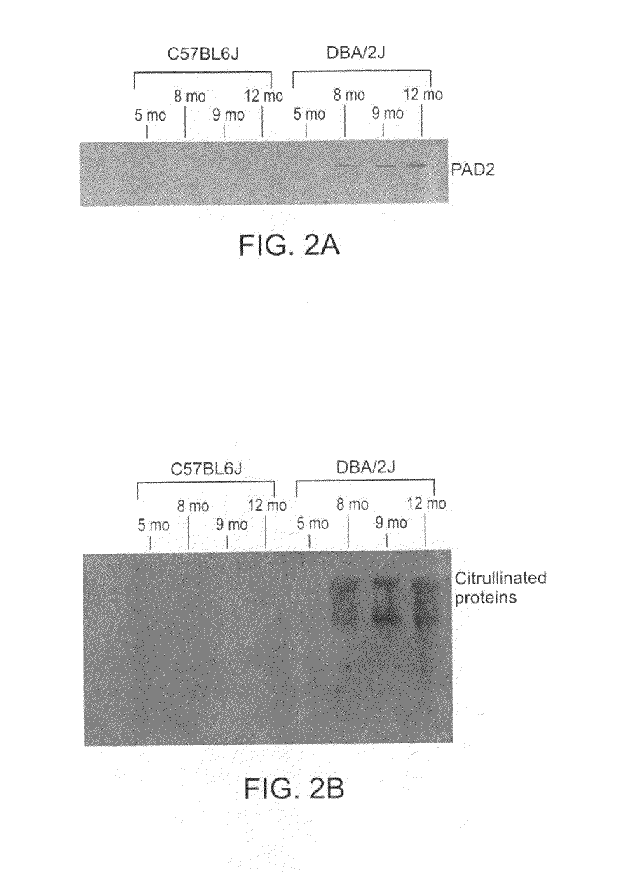

Western analyses detected PAD2 and citrullination in the optic nerve of 8-12 month old DBA/2J mice, but not in DBA/2J mice at 5 months of age, nor in 5-12 month old control C57BL6J mice which do not exhibit increased IOP (FIG. 2A, 2B). These observations show that the glaucomatous DBA/2J mouse exhibits parallel features of the optic nerve damage found in human POAG.

### Increased Citrullination and Decreased Methylation in Glaucomatous Optic Nerve

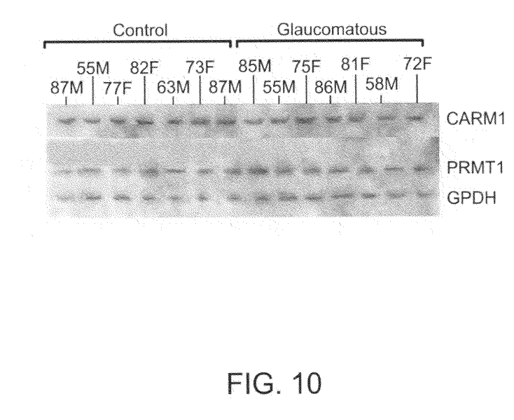

Western analyses of POAG optic nerve showed increased PAD2, increased citrullination but decreased protein arginyl methylation relative to the normal controls (FIG. 1B, 1C, 1D). Decreased arginyl methylation (FIG. 1D) concomitant with increased citrullination (FIG. 1C) is consistent with the conversion of arginine to citrulline prior to methylation (FIG. 1B). However, to determine if the decreased levels of methylated proteins in POAG optic nerve could be due to down-regulation of protein methylation activities, Western analyses for both protein arginine N-methyltransferase 1 (PRMTI) and coactivator-associated arginine methyltransferase 1 (CARM 1) was performed. Normalized to glyceraldehydes phosphate dehydrogenase (GPDH), the expression levels of CARM1 and PRMTI appear to be comparable in control and POAG optic nerve (FIG. 10). We also assayed methyltransferase activity in S-adenosyl-L-methionine (S-AdoMet) depleted crude tissue extracts using radiolabeled S-AdoMet and found essentially identical activities (40075±1515 cpm and 40615±2061 cpm) in control and glaucomatous optic nerve tissue (three independent experiments). Decreased methylation could also be due to demethylimination of methylated protein arginines and this possibility cannot be ruled out. Although protein demethylating activity of PAD4 (a nuclear protein) was recently discovered and reconciled as demethylimination (Cuthbert et al., Cell, 118:545-533 (2004); Wang et al., Science, 306:279-283 (2004)), no cytosolic deiminase has yet been shown to demethyliminate. PAD4 was not identified in the optic nerve tissue by proteomic ananlysis. In any event, the observed citrullination is due to increased deiminase activity and the decreased level of methylated arginine is not due to lack of protein methyltransferease (FIG. 10).

### Immunohistochemical Localization of PAD2 and Citrullinated Proteins

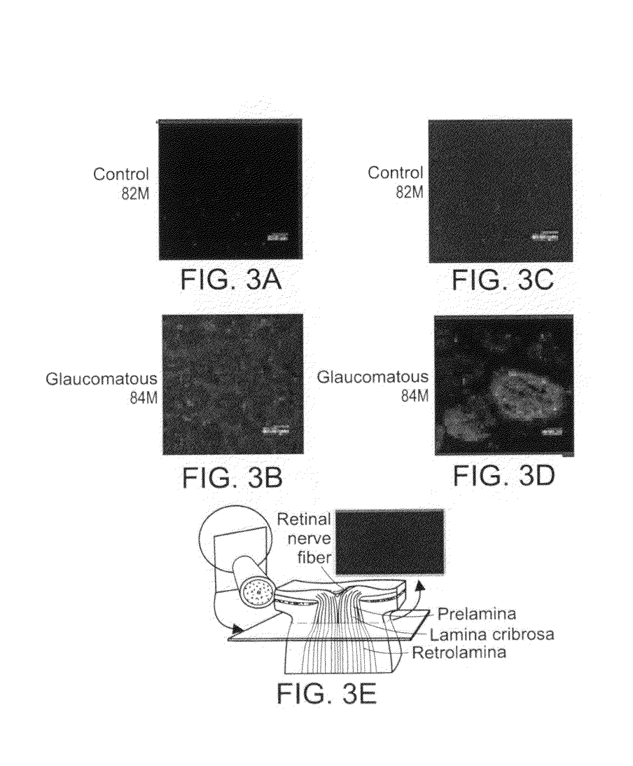

Immunohistochemical analyses showed localization of PAD2 in the lamina cribrosa region of POAG optic nerve (FIG. 3A, 3B) along with citrullinated proteins (FIG. 3C, 3D). The lamina cribrosa region of the optic nerve is shown schematically in FIG. 3E. Identically treated control and glaucomatous optic nerve showed clear differences in citrullinated protein content in the lamina cribrosa region (FIG. 3C, 3D). Normal control optic nerve exhibited much less immunohistochemical reactivity for citrullinated proteins.

Consistent with elevated levels of PAD2 in POAG by Western analyses (FIG. 1B) citrullinated proteins were observed throughout the optic nerve of POAG lamina cribrosa region, control optic nerve exhibited much less immunoreactivity for citrullinated proteins. For immunohistochemical detection of citrulline, the tissue was treated with 2,3-butanedione monooxime and antipyrine in a strong acid atmosphere. This enables chemical modification of citrulline into ureido groups and ensures detection of citrulline-containing proteins regardless of neighboring amino acid sequences (Senshu T., et al., Anal. Biochem., 203:94-100 (1992)). Processing steps in a strong acid environment and antipyrine makes nuclear staining (TOPRO3) less pronounced. However, identically treated control and glaucomatous tissue showed clear difference in citrullinated protein content in the lamina cribosa region (FIGS. 3A-3D).

### Identification of Citrullinated Proteins in POAG Optic Nerve

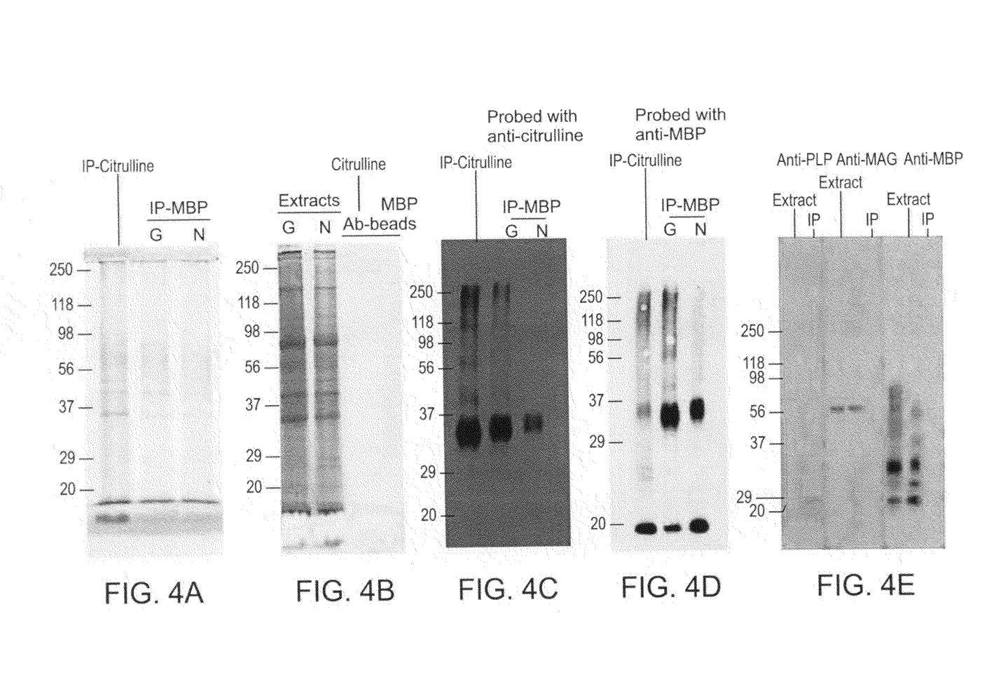

Proteins in POAG optic nerve were immunoprecipitated with anti-citrulline antibody for protein identification (FIG. 4A, 4B). The most intense citrulline immunoreactive component in these immunoprecipitations (IPs) was identified by mass spectrometric and Western analysis as myelin basic protein (MBP), indicating that this is a major citrullinated protein in POAG optic nerve (FIG. 4C, 4D). To confirm this finding, anti-MBP was also used to immunoprecipitate proteins from normal and POAG optic nerve and subsequently probed with anti-citrulline antibody. More citrullinated MBP was observed in POAG than in normal optic nerve extracts (FIG. 4A-4E). Mass spectrometric and Western analyses of anti-citrulline immunoprecipitation products also detected citrullinated myelin proteolipid protein and myelin associated glycoprotein in POAG optic nerve (FIG. 4E). Mass spectrometry also detected myelin P0 protein and myelin oligodendrocyte protein in the anti-citrulline IP. Other proteins identified in the anti-citrulline IP are listed in Table 2.

### Pressure Upregulates PAD2 Expression In Vivo and In Vitro

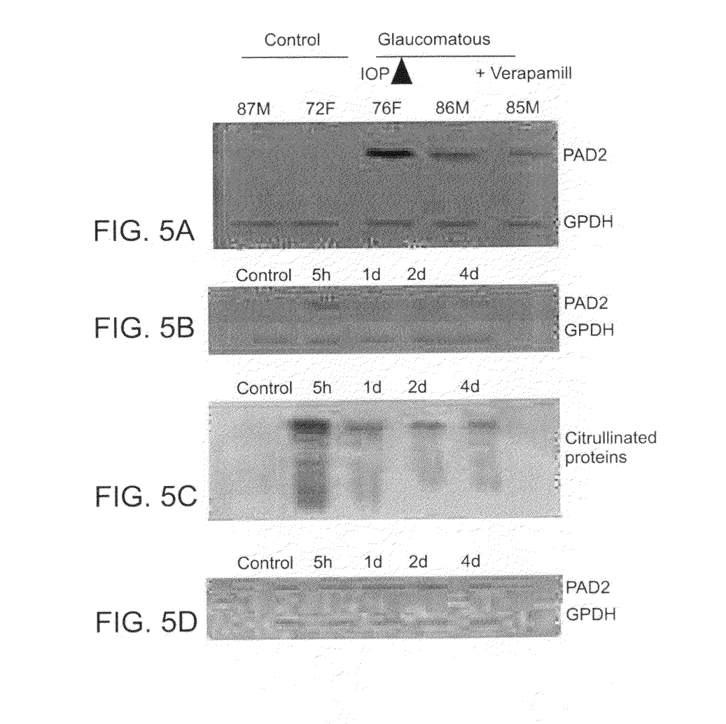

Apparent high levels of PAD2 were observed in donors with elevated IOP (FIG. 5A). Optic nerve from a 76 year old female POAG donor with high IOP but no surgical or pharmacological intervention, and without head or eye injury from a fatal automobile accident, was found to exhibit a very high level of PAD2. Other donor eyes that had undergone either surgical or combined surgical and pharmacological intervention to relieve elevated IOP were also analyzed for PAD2. Notably, we observed that POAG optic nerve PAD2 remained detectable after surgical or pharmacological intervention and after the IOP returned to normal. Results from two such glaucomatous donors (86M and 85M) subjected to trabeculectomy with the 85M donor also receiving verapamil (a calcium modulator) are presented in FIG. 5A. Although both (86M and 85M) show lower levels of optic nerve PAD2 compared to those without intervention (76F), their PAD2 level is still high compared to normal controls. In the verapamil treated eye (85M), the PAD2 level appeared lower than in the other POAG eyes with or without intervention (86M and 76F).

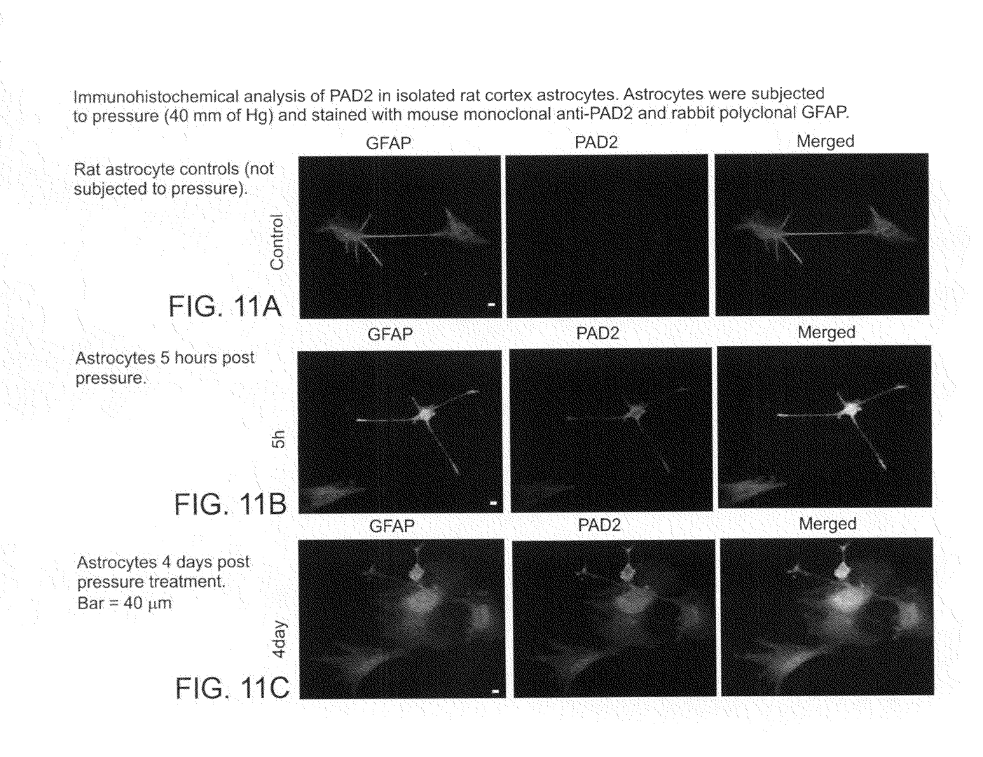

To determine whether pressure induces PAD2 and subsequent citrullination, primary rat cortex astrocyte cultures were subjected to an increase in pressure by 40 mm of Hg for 5 hours and then restored to atmospheric pressure. The short term elevated pressure led to increased PAD2 in astrocytes that was still detectable after four days at atmospheric pressure by Western analysis (FIG. 5B) and immunohistochemistry (FIGS. 11A-11C). Increased citrullination was also observed in the astrocytes concomitant with pressure treatment and remained detectable after four days at atmospheric pressure (FIG. 11C). In contrast to pressure-induced changes in PAD2 protein expression, by Northern analysis PAD2 mRNA levels did not significantly change in astrocytes subjected to pressure (FIG. 5D). These observations were replicated in astrocytes derived from a 7 year old human optic nerve head.

### Translational Control of PAD2 Overexpression

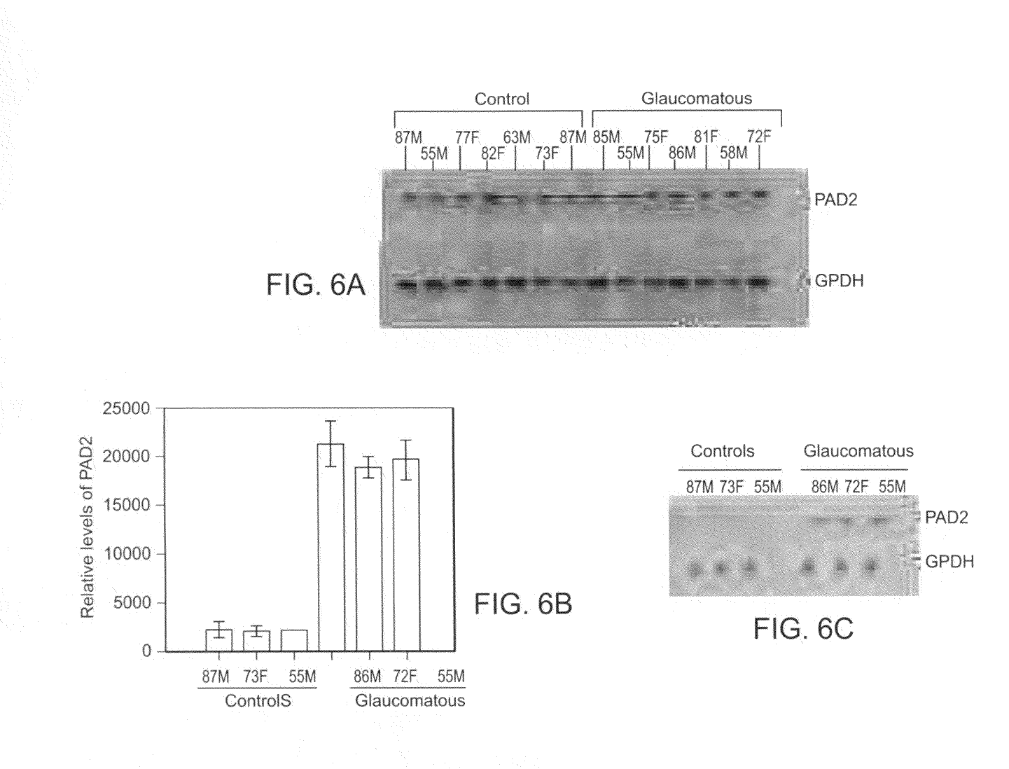

To further probe whether increased PAD2 expression in vivo is due to increased PAD2 mRNA, total RNA from normal human and POAG optic nerve were subjected to Northern analysis. The amount of the PAD2 transcript normalized to that of GPDH was found to be very similar between 7 control and 7 glaucomatous donors (FIG. 6A), suggesting PAD2 over expression in POAG optic nerve may be translationally regulated. Additional experiments supporting translational control of PAD2 expression were performed with normal and POAG optic nerve extracts depleted of both polyadenylated RNA and the PAD2 and GPDH proteins (FIG. 6B, 6C). These depleted extracts lack translation capability without exogenous mRNA. Upon addition of exogenous polyadenylated RNA to the depleted extracts, a large increase in PAD2 expression (relative to GPDH) was observed in the POAG extracts but not in the control extracts (FIG. 6B, 6C).

Identical gels with fresh optic nerve extracts depleted of total mRNA (using oligo-dT column) and PAD2, GPDH proteins (antibody columns) were introduced with equal amounts of mRNA for PAD 2 and GPDH with cold or S-35 labelled methionine. Immuno-reactive bands for PAD 2 and GPDH identified from Western blot of cold gel were compared with radioactive gel, excised and counted on a scintillation counter (FIGS. 6B, 6C). Control of PAD 2 overexpression at the level of translation in POAG is supported by the fact that with equal amount of addition of polyadenylated RNA more than nine fold increase in PAD 2 relative to GPDH was observed in glaucomatous than in control optic nerve extracts depleted of poly adenylated RNA, PAD 2 and GPDH (FIGS. 6B, 6C).

Down-Regulation of Citrullination with PAD2 shRNA

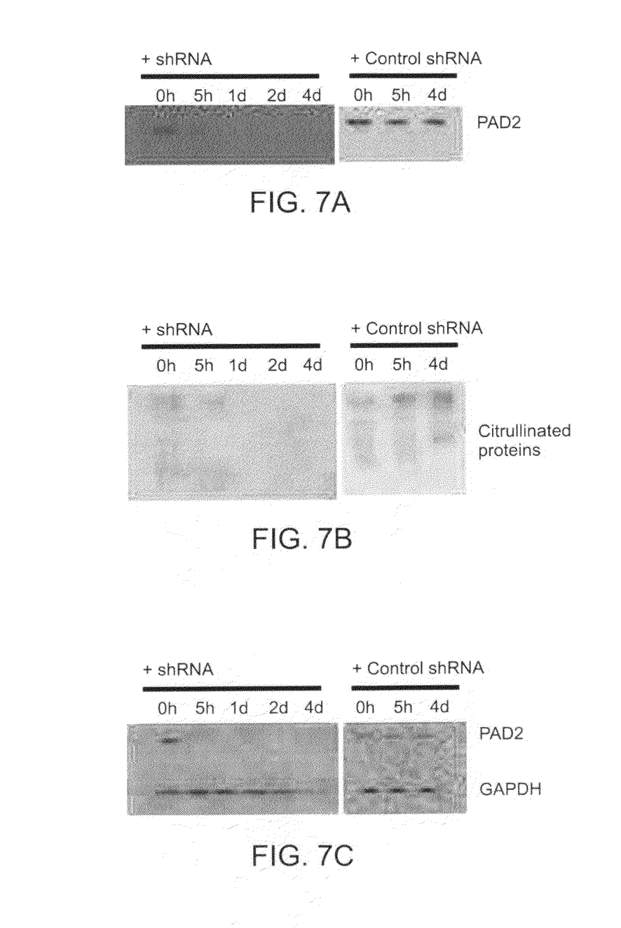

Increased PAD2 and citrullination were observed in astrocytes subjected to pressure even after restoration of atmospheric pressure (FIG. 5). As a possible approach to reducing pressure-induced citrullination, the effect of lowering PAD2 mRNA in vitro in astrocytes was tested. Primary culture astrocytes were subjected to pressure and transfected with shRNA immediately after they were brought to atmospheric pressure. It was found that astrocytes treated with a PAD2 specific shRNA (but not with a non-specific shRNA) exhibited reduced PAD2 expression (FIG. 7A) and reduced citrullination (FIG. 7B) as a consequence of degradation of the mRNA transcript (FIG. 7C). Although some residual citrullination was observed, PAD2 mRNA was completely removed by shRNA within the sensitivity of detection (FIG. 7C).

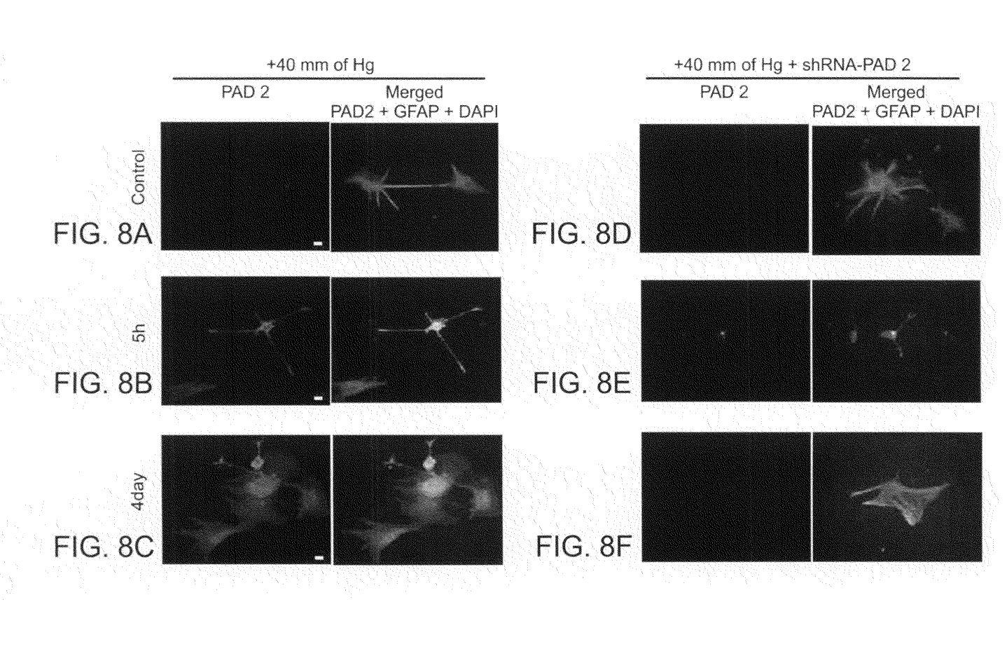

The primary astrocytes were subjected to increased pressure and transfected with shRNA on plates immediately after they were brought to atmospheric pressure. This regime was used to model astrocytes as to what could possibly be applied to eyes. Once the pressure is brought to normal by surgical intervention, the eyes could be amenable to siRNA or shRNA treatment either immediately or after an incubation period. The immediate shRNA treatment considering future ease in application while evaluating in animal models was selected. Although some residual citrullination was observed, PAD 2 mRNA was completely removed by shRNA within the sensitivity level of our detection (FIG. 7C). Differences in cell morphology due to this reduction in mRNA was not observed (FIGS. 8A-8F). Control rat astrocytes not subjected to pressure does not stain with PAD 2 antibody but shows GFAP immunoreactivity (FIGS. 8A, 8D). The cells after 5 hours pressure treatment (40 mm of Hg) were brought to atmospheric pressure and subjected to shRNA treatment. FIGS. 8A-8D show astrocytes with indicated times of incubation at atmospheric pressure (5 hours or 4 days). Immunohistochemical analysis showed PAD 2 reduced in isolated astrocytes treated with shRNA (FIG. 8E, 8F) as compared to untreated group (FIGS. 8B, 8C).

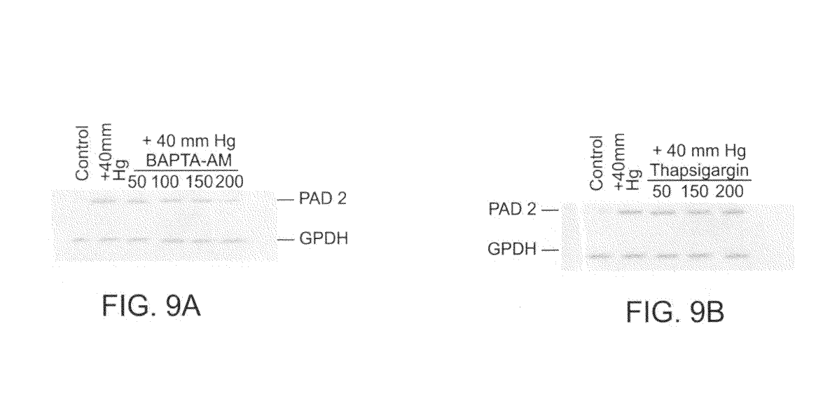

Modulation of calcium leads to PAD 2 level changes in astrocytes. The cultured astrocytes were subjected to a pressure of 40 mm Fig and subjected to a decrease in calcium concentration by chelating agent BAPTA-AM (50-200 nM) or increased intracellular calcium using Thapsigargin (50-200 nM). As shown in FIGS. 9A-9B decrease in intracellular concentration using BAPTA-AM (FIG. 9A) reduces expression of PAD 2 protein. Intracellular calcium increase using Thapsigargin (FIG. 9B) seems not to show a great increase in PAD 2 expression, however the level of PAD 2 remains elevated and it is not completely possible to determine whether this is the highest level of PAD 2 achievable under these conditions. It is important to note that these reagents have been used at sublethal doses, that is, at a concentration where they do not trigger apoptosis.

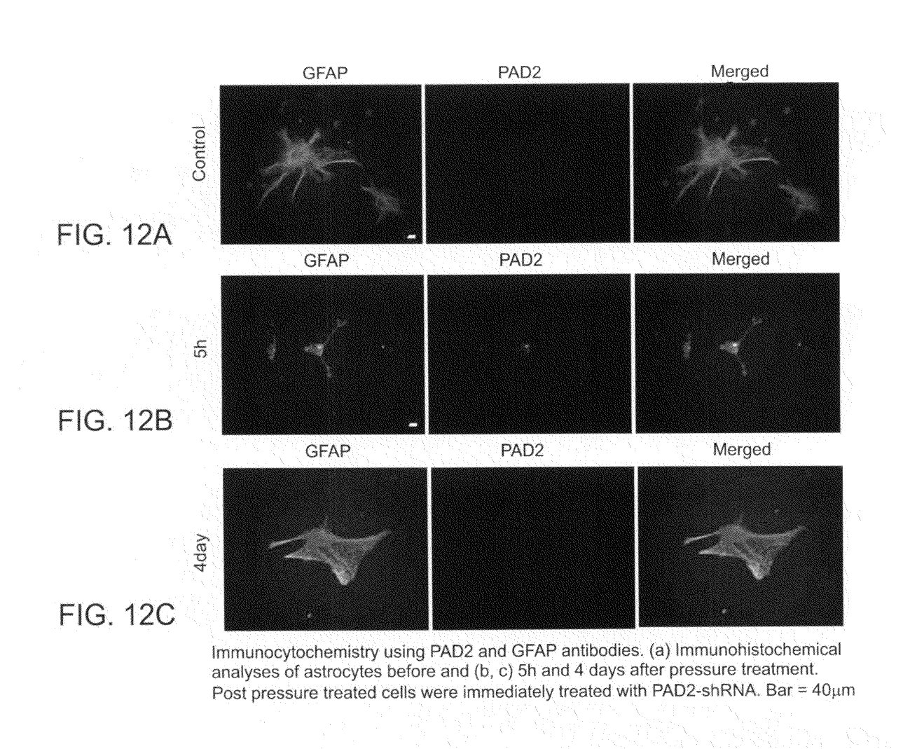

Differences in cell morphology due to this reduction in mRNA were not observed, although immunohistochemical analysis showed that PAD2 was reduced in astrocytes treated with shRNA (FIGS. 12B, 12C) as compared to pressure treated group without shRNA (FIGS. 11B, 11C).

### Additional Examples of PAD2 shRNA

shRNA can be delivered in a variety of vectors (e.g., lentiviral vector, adenoviral vector). For example, lentiviral vectors have been shown to confer long term expression in optic nerve with high (>80%) efficiency (Harvey, A. R., et al. Mol. Cell Neurosci., 21:141-157 (2002); van Adel, B. A., et al., Hum. Gene Ther., 14:103-115 (2003)). Methods which evaluate constructs in vitro in primary optic nerve cultures that are well-established practice (Hannon and Conklin, Methods Mol. Biol., 257:255-266 (2004)) and in vivo using one or more appropriate animal models (e.g., DBA/2J mice) can be used to assess the shRNA. shRNA for PAD 2 (OpenBiosystems, cat. #RHS 1764-9214220) can be cloned in pShag Magic version 2.0 (OpenBiosystems) that will express inhibitor hairpin. Additionally, systems such as the BLOCK-iT™ Designer (Invitrogen corporation) that uses a proprietary algorithm to design shRNA with the latest research data to optimize for promoter requirements and stem-loop structure can be used. The following five sequences have been identified, in which the start position in PAD 2 gene and percent GC content is shown below.

| No. | Start | Target DNA sequence | % GC |
|---|---|---|---|
| 1 | 653 | GGATACGAGATAGTTCTGTACATTT | 36.0 |
| | | (SEQ ID NO: 8) | |
| 2 | 914 | CCCATCTTCACGGACACCGTGATAT | 52.0 |
| | | (SEQ ID NO: 9) | |
| 3 | 1179 | CCCGAGATGGAAACCTAAAGGACTT | 48.0 |
| | | (SEQ ID NO: 10) | |
| 4 | 1623 | GGATGAGCAGCAAGCGAATCACCAT | 52.0 |
| | | (SEQ ID NO: 11) | |
| 5 | 1811 | GCCTTCTTCCCAAACATGGTGAACA | 48.0 |
| | | (SEQ ID NO: 12) | |

An example of a method to assess shRNA is as follows. At 2-4 weeks of age, DBA/2J mice are anesthetized and 1-2 μl is injected into the intravitreal region of the right eye using a pulled capillary pipette (7-20 gm tip diameter) attached to a 10 R1 Hamilton syringe as per the published protocols (Harvey, A. R., et al. Mol. Cell Neurosci., 21:141-157 (2002)). The left eye is used as an uninjected control. Injections (10⁶-10⁹ transduction units of lentiviral vectors, contained in approximately 1 μl vehicle) are performed. An empty vector is used as a control.

In most DBA/2J mice the IOP is elevated around 8 months of age. At 3, 6, 9, 12 and 18 months, IOP is measured using, for example, a method adapted from John, S. W., et al., Invest. Opthalmol. Vis. Sci., 38:249-253 (1997). Mice are then sacrificed and eyes are examined by Western and Northern blot analysis to determine PAD2/GPDH levels, immunohistochemistry to determine PAD 2 distribution, and the optic nerve is examined histopathologically to determine the presence and severity of optic nerve damage.

Appropriate shRNA for use in the methods describer herein will exhibit a decrease in PAD 2 and citrullination by down-regulation of PAD 2 message in DBA/2J mice. PAD 2 mRNA can be reduced by more than about 70% in the optic nerve of DBA/2J mice infected with shRNA for a prolonged period. DBA/2J mice treated with such shRNA will exhibit a less severe glaucoma phenotype with reduced progression rate of optic nerve degeneration than DBA/2J mice treated with a control vector. The demonstration of lack of citrullinated proteins and lack of aberrant localization of select citrullinated proteins upon down regulation of PAD 2 can also be to assess appropriate shRNA for use in the methods of the present invention.

Single or multiple injections of siRNA at every three-month intervals can be used. A variety of promoters can also be used. Additional sequences for PAD 2 coding region (NM 007365) using a program provided by Ambion corporation includes:

| Position in gene sequence of NM 007365: 290; | |
|---|---|
| GC content: 42.9% | |
| Sense strand siRNA: | |
| GGUCACCGUCAACUACUAUtt | (SEQ ID NO: 13) |
| Antisense strand siRNA: | |
| AUAGUAGUUGACGGUGACCtt | (SEQ ID NO: 14) |
| Position in gene sequence of NM 007365: 416; | |
| GC content: 42.9% | |
| Sense strand siRNA: | |
| GAACAACCCAAAGAAGGCAtt | (SEQ ID NO: 15) |
| Antisense strand siRNA: | |
| UGCCUUCUUUGGGUUGUUCtt | (SEQ ID NO: 16) |
| and, | |
| Position in gene sequence of NM 007365: 708; | |
| GC content: 47.6% | |
| Sense strand siRNA: | |
| CGCUAUAUCCACAUCCUGGtt | (SEQ ID NO: 17) |
| Antisense strand siRNA: | |
| CCAGGAUGUGGAUAUAGCGtt | (SEQ ID NO: 18) |

An example of an appropriate dosage of siRNA is 15 nmole siRNA. Optionally, morpholino oligonucleotides can be used for these sequences as a stand by measure. siRNA with a 3′TT preferred end structure (AMBION) can also be used in the methods of the present invention. This program scans the gene sequence for AA dinucleotides and a standard 21 base target and the corresponding sense and antisense siRNA oligonucleotides provided. G/C content is calculated, siRNAs with lower G/C content (30-50%) are more active than those with higher G/C content. Both Invitrogen and Ambion programs allow one to limit siRNA choices by maximum G/C content and the designed shRNA described herein have more than 30 but less than 50 percent GC content. Invitrogen and Ambion guarantee that one out of three construct using their respective programs will be effective in over 70 percent message down-regulation.

### Discussion

Classical proteomic methods initially detected PAD2 in the optic nerve of glaucomatous but not normal human donors. Subsequently, PAD2 was found to be uniquely associated with glaucomatous human optic nerve by Western and immunohistochemical analyses of additional POAG and normal donor tissues. Western analysis also demonstrated the presence of PAD2 in optic nerve from the DBA/2J glaucomatous mouse at ages 8-12 months, but not in younger DBA/2J mice that do not exhibit elevated IOP nor in optic nerve from control C57BL6J mice. Proteomic analyses identified many other proteins in human optic nerve (Table 1), however the significance of other proteins detected only in glaucomatous optic nerve remains to be determined.

PAD2 converts arginine to citrulline and observed increased protein citrullination and decreased protein arginyl methylation in POAG optic nerve was observed as described herein. Recently, PAD2 directed citrullination was associated with kainite-induced neurodegeneration in rat brain (Asaga, H., et al., Neurosci. Lett., 326:129-132 (2002); Asaga, H., et al., Neurosci. Lett., 299:5-8 (2001)). PAD2 predominantly occurs in neuronal tissues (Moscarello, M. A., et al., J. Neurochem., 81:335-343 (2002)) however five protein deiminases have been identified in a variety of tissues, including protein deiminases 1, 2, 3 and 6 which are cytosolic and protein deiminase 4 which exhibits nuclear localization (Nakashima, K., et al., J. Biol. Chem., 277:49562-49568 (2002)). PAD4 was recently found to catalyze reverse methylation or demethylination as well as deimination of proteins (Cuthbert, G. L., et al., Cell., 118:545-553 (2004); Wang, Y., et al., Science, 306:279-283 (2004)). PADs have been implicated in demyelinating diseases (Moscarello, M. A., et al., J. Neurochem., 81:335-343 (2002)) and citrullination has been implicated in diseases such as autoimmune rheumatoid arthritis (Scofield, R. H., et al., Lancet, 363:1544-1546 (2004)), multiple sclerosis (Moscarello, M. A., et al., J. Neurochem., 81:335-343 (2002)) and amyotrophic lateral sclerosis (Chou, S. M., et al., J. Neurol. Sci., 139 Suppl:16-26 (1996)).

The consequences of citrullination are many and varied. Notably, myelin contains several arginine-rich proteins that are susceptible to citrullination (Carelli, V., et al., Neurochem. Int., 40:573-584 (2002)), including MBP which was detected herein as a major citrullinated protein in POAG optic nerve. MBP is one of the most abundant proteins of the myelin sheath and functions in maintaining the stability of the sheath (Kursula, P., et al., J. Neurochem., 73:53-55 (1999)). Citrullinated MBP exhibits altered properties relative to the unmodified protein, including a lower net positive charge, which disrupts its tertiary structure and ability to interact with lipids and maintain a compact myelin sheath (Boggs, J. M., et al., Biochem., 36:5065-5071 (1997); Pritzker, L. B., et al., Biochem., 39:5382-5388 (2000)). Citrullination also decreases the ability of MBP to aggregate large unilamellar vesicles (LUVs) (Boggs, J. M., et al., Biochem., 36:5065-5071 (1997)), a process important for adhesion between intracellular surfaces of myelin. Citrullinated MBP exhibits increased susceptibility to cathepsin D proteolysis, which may generate immunodominant peptides leading to sensitization of T-cells for the autoimmune response in demyelinating diseases (Pritzker, L. B., et al., Biochem., 39:5382-5388 (2000)). Citrullination also appears to inhibit cell proliferation, leading to cell cycle arrest and apoptosis (Gong, H., et al., Leukemia, 14:826-829 (2000); Gong, H., et al., Biochem. Biophys. Res. Commun., 261:10-14 (1999)). Such mechanisms may all play a role in glaucomatous neuropathy. The presence of multiple citrullinated proteins in POAG optic nerve, including MBP, myelin proteolipid protein and myelin associated glycoprotein among others, would appear likely to disrupt myelination. Citrullination of optic nerve head matrix proteins may weaken their anchorage and overall weakness at the level of optic nerve head. It is likely that citrullination causes changes in the dynamics of myelin components and also may cause disruption of the optic nerve head matrix protein framework that may initiate or contribute to glaucomatous neuropathy.

A variety of factors trigger PAD2 expression. The results described herein (FIG. 5A-5D) demonstrate that pressure induces PAD2 expression in vitro in astrocytes, and others have shown in astrocytes that hypoxia induces PAD2 expression, citrullination and elevated intracellular calcium concentration (Sambandam, T., et al., Biochem. Biophys. Res. Commun., 325:1324-1329 (2004); Osborne, N. N., et al., Surv. Ophthalmol., 43, Suppl. 1:S102-S108 (1999)). Calcium imbalance has been implicated in eliciting PAD2 activity (Asaga, H., et al., Neurosci. Lett., 299:5-8 (2001)), and perhaps calcium influences the increased PAD2 observed in myelinating immature oligodendrocytes (Akiyama, K., et al., Neurosci. Lett., 274:53-55 (1999)). Increased IOP in glaucoma often is associated with influx of calcium (e.g., from ischemia), resulting in increased intracellular calcium (Osborne, N. N., et al., Surv. Ophthalmol., 43, Suppl. 1:S102-S108 (1999)). Notably in myelin, calcium concentration plays an important role in modulating a number of protein interactions (Kursula, P., et al., J. Neurochem., 73:1724-1732 (1999); Marta, C. B., et al., J. Neurosci. Res., 69:488-496 (2002)) including for example MBP interaction with calmodulin, which citrullination can disrupt (Libich, D. S., et al., Protein Sci., 12:1507-1521 (2003)). In POAG, events triggered by intraocular pressure, including fluctuations in optic nerve intracellular calcium concentration, may increase the level of PAD2 and citrullination.

The observations proved herein are consistent with post-transcriptional control of PAD2 expression. In a preliminary analyses, optic nerve derived RNA (pooled from two donors each, control and glaucomatous) used in a microarray analysis revealed changes in mRNA levels for 1923 proteins (GSE2387: NCBI GEO database) between control and glaucomatous optic nerve tissue, however, PAD2 was not among them. Optic nerve PAD2 mRNA levels appear to be very similar between control and glaucomatous donors in vivo (FIG. 6A) and between pressure treated and untreated astrocytes in vitro (FIG. 5D). However, glaucomatous optic nerve extracts depleted of polyadenylated RNA, PAD2 and GPDH, exhibited a significant increase in PAD2 expression (relative to GPDH) upon addition of equal amounts of polyadenylated RNA with no comparable increase in control extracts (FIG. 6B, 6C). This in vitro data indicates that the over expression of PAD2 in glaucomatous tissue is primarily controlled at the translational level. However, in vivo, a lower normal steady state expression level could result from an increased degradation rate as well as from a decrease in the rate of translation. As shown herein, in vitro targeted degradation of PAD2 mRNA with shRNA in pressure treated astrocytes leads to a decrease in PAD2 and citrullination. The present results implicate optic nerve PAD2 directed citrullination in glaucoma pathogenesis.

### Example 2

### PAD2 Assay and Inhibitors in Plant Extracts

### PAD Activity Assay

For determination of PAD activity, HEK cells expressing PAD2 were ruptured by sonication, and the entire lysates were incubated with benzoyl-L-arginine ethyl ester (BAEE) or benzoyl-L-arginine (BzArg) as a substrate following standard protocols (Watanbe et. al., Biochim. Biophys. Acta, 966:375-383 (1988)). One unit was defined as the amount of enzyme catalyzing the formation of 1 mmol of citrulline derivative in 1 h at 50° C. Protein concentrations were determined by the method of Bradford (Bradford, M. M. Anal Biochem., 72:248-254 (1976)) using bovine serum albumin as a standard. For estimation of inhibition by plant extracts 1 microliter of plant extract was added to a total of 100 microliter assay mixture (less than 2% volume).

Benzoyl-L-arginine ethyl ester (BAEE): Catalog #: B4500-10G (SIGMA-ALDRICH); Catalog #: B4500-25G (SIGMA-ALDRICH) or Benzoyl-L-arginine (BzArg): Catalog #: IC15482983 (VWR International) Asaga H., et al., J. Leukoc. Biol., 70(1):46-51 (2001).

### Preparation of Plant Extracts

### Olive Leaf Extract

Olive leaves were procured and about 5 g of olive leaves were extracted with 2-5 ml of chloroform-methanol (97:3) and extracted principles were dried in a speedVac and resuspended in 125 mM Tris-Cl buffer pH 8.0 containing 100 mM NaCl, a blank buffer was used to confirm that buffer alone did not affect the enzymatic activity. The extract was used to test the inhibitory activity in the PAD assay described above.

### Vitex Agnus Cactus

The cactus stem (50 g) was extracted with 10 ml of n-propanol-toluene-glacial acetic acid-water (25:20:10:10) at room temperature. The extractant was dried in a speedVac and suspended in 50 mM Tris-Cl pH 8.0 containing 125 mM NaCl, a blank buffer was used to confirm that buffer alone did not affect the enzymatic activity. Once microliter of the extract was used to determine the inhibitory activity in the PAD assay described above.

### Vinca Rosea Extract

Vinca leaves were procured and about 5 g of leaves were extracted with 2-5 ml of chloroform-methanol (97:3) and extracted principles were dried in a speedVac and resuspended in 125 mM Tris-Cl buffer pH 8.0 containing 100 mM NaCl. The extract was used to test the inhibitory activity in the PAD assay described above. Usually this extract did not show any inhibitory activity when 1 microliter fractions were used.

### Results

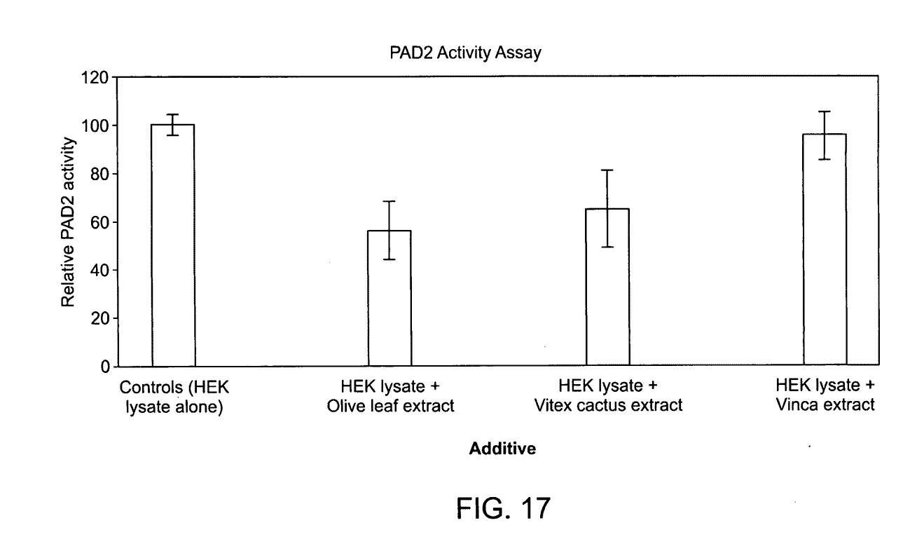

As shown in FIG. 17, olive leaves extract and cactus extract resulted in significant reduction of the activity, compared to control or vinca rosea extract which did not. Control was lysate of HEK cells without any addition. These results indicate that olive leaves and vitex agnus cactus have active constituents that affect PAD2 activity determined by the above assays.

The entire teachings of all references cited herein are incorporated herein by reference.

TABLE 1

| | | | |
|---|---|---|---|
| Accession | | Peptide | |
| Number^{a} | Protein | Matches | Frequency^{b} |

Proteins identified only in glaucomatous optic nerve

| | | | |
|---|---|---|---|
| P14618 | Pyruvate kinase, M1 isozyme | 9 | 8 |
| P16152 | NADPH-dependent carbonyl reductase 1 | 7 | 7 |
| P02511 | Alpha crystallin B chain | 5 | 7 |
| P61204 | ADP-ribosylation factor 3 | 4 | 4 |
| P40926 | Malate dehydrogenase, mitochondrial precursor | 3 | 4 |
| Q9Y2J8 | Protein-arginine deiminase type II | 3 | 4 |
| P00505 | Aspartate aminotransferase, mitochondrial | 2 | 4 |
| P01842 | Ig lambda chain C regions | 2 | 4 |
| P13591 | Neural cell adhesion molecule 1, 140 kDa isoform precurs | 2 | 4 |
| P68104 | Elongation factor 1-alpha 1 | 2 | 4 |
| P00387 | NADH-cytochrome b5 reductase | 3 | 3 |
| P02808 | Slatherin precursor | 2 | 3 |
| P45880 | Voltage-dependent anion-selective channel protein 2 | 2 | 3 |
| P01876 | Ig alpha-1 chain C region | 5 | 2 |
| P02023 | Hemoglobin beta chain | 5 | 2 |
| P21333 | Filamin A | 5 | 2 |
| P05091 | Aldehyde dehydrogenase, mitochondrial precursor | 4 | 2 |
| P33778 | Histone H2B.f | 4 | 2 |
| P50395 | Rab GDP dissociation inhibitor beta | 4 | 2 |
| P31946 | 14-3-3 protein beta/alpha | 3 | 2 |
| Q14697 | Neutral alpha-glucosidase AB precursor | 3 | 2 |
| P02689 | Myelin P2 protein | 2 | 2 |
| P10809 | 60 kDa heat shock protein, mitochondrial precursor | 2 | 2 |
| P12273 | Prolactin-inducible protein precursor | 2 | 2 |
| P17174 | Aspartate aminotransferase, cytoplasmic | 2 | 2 |
| P34932 | Heat shock 70 kDa protein 4 | 2 | 2 |
| P38646 | Stress-70 protein, mitochondrial precursor | 2 | 2 |
| P53674 | Beta crystallin B1 | 2 | 2 |
| P60891 | Ribose-phosphate pyrophosphokinase I | 2 | 2 |
| Q13938 | Calcyphosine | 2 | 2 |
| Q16378 | Proline-rich protein 4 precursor | 2 | 2 |
| Q9BPU6 | Dihydropyrimidinase related protein-5 | 2 | 2 |
| P14786 | Pyruvate kinase, M2 isozyme | 12 | 1 |
| P48666 | Keratin, type II cytoskeletal 6C | 9 | 1 |
| P04745 | Alpha-amylase | 7 | 1 |
| Q9NP55 | Protein Plunc precursor | 5 | 1 |
| P00751 | Complement factor B precursor | 4 | 1 |
| P13646 | Keratin, type I cytoskeletal 13 | 4 | 1 |
| P00367 | Glutamate dehydrogenase 1, mitochondrial precursor | 3 | 1 |
| P01877 | Ig alpha-2 chain C region | 3 | 1 |
| P08603 | Complement factor H precursor | 3 | 1 |
| P11217 | Glycogen phosphorylase, muscle form | 3 | 1 |
| P17317 | Histone H2A.z | 3 | 1 |
| P34931 | Heat shock 70 kDa protein 1-HOM | 3 | 1 |
| Q9NZT1 | Calmodulin-like protein 5 | 3 | 1 |
| Q9Y281 | Cofilin, muscle isoform | 3 | 1 |
| Q9Y490 | Talin 1 | 3 | 1 |
| O75891 | 10-formylletrahydrofolate dehydrogenase | 2 | 1 |
| P00488 | Coagulation factor XIII A chain precursor | 2 | 1 |
| P00491 | Purine nucleoside phosphorylase | 2 | 1 |
| P00568 | Adenylate kinase isoenzyme 1 | 2 | 1 |
| P01833 | Polymeric-immunoglobulin receptor precursor | 2 | 1 |
| P02489 | Alpha crystaliin A chain | 2 | 1 |
| P02814 | Proline-rich protein 3 precursor | 2 | 1 |
| P23527 | Histone H2B.n | 2 | 1 |
| P30044 | Peroxiredoxin 5 | 2 | 1 |
| P31944 | Caspase-14 precursor | 2 | 1 |
| P35558 | Phosphoenolpyruvate carboxykinase | 2 | 1 |
| P46940 | Ras GTPase-activating-like protein IQGAP1 | 2 | 1 |
| P47929 | Galectin-7 | 2 | 1 |
| P51148 | Ras-related protein Rab-5C | 2 | 1 |
| P55786 | Puromycin-sensitive aminopeptidase | 2 | 1 |
| P62158 | Calmodulin | 2 | 1 |
| P81605 | Dermcidin precursor | 2 | 1 |
| Q16778 | Histone H2B.q | 2 | 1 |
| Q16836 | Short chain 3-hydroxyacyl-CoA dehydrogenase, mitochon | 2 | 1 |
| Q9BXN1 | Asporin precursor | 2 | 1 |
| Q9Y4W6 | AFG3-like protein 2 | 2 | 1 |

Proteins identified only in control optic nerve

| | | | |
|---|---|---|---|
| Q04917 | 14-3-3 protein eta | 2 | 1 |
| Q9Y4L1 | 150 kDa oxygen-regulated-protein precursor | 6 | 1 |
| P02765 | Alpha-2-HS-glycoprotein precursor | 2 | 1 |
| P55087 | Aquaporin 4 | 2 | 1 |
| O43852 | Calumenin precursor | 4 | 1 |
| Q13740 | CD166 antigen precursor | 2 | 1 |
| P09622 | Dihydrolipoyl dehydrogenase, mitochondrial precursor | 2 | 2 |
| P15311 | Ezrin | 5 | 1 |
| P52907 | F-actin capping protein alpha-1 subunit | 3 | 1 |
| P09972 | Fructose-bisphosphate aldolase C | 3 | 4 |
| P28161 | Glutathione S-transferase Mu 2 | 2 | 1 |
| P11142 | Heat shock cognate 71 kDa protein | 3 | 1 |
| Q14525 | Keratin, type I cuticular HA3-II | 5 | 1 |
| P05783 | Keratin, type I cytoskeletal 18 | 2 | 1 |
| O93532 | Keratin, type II cytoskeletal cochleal | 2 | 3 |
| P02788 | Lactotransferrin precursor | 2 | 1 |
| P15586 | N-acetylglucosamine-6-sulfatase precursor | 2 | 1 |
| P13590 | Neural cell adhesion molecule 1, 180 kDa isoform precurs | 2 | 1 |
| P59665 | Neutrophil defensin 1 precursor | 2 | 1 |
| P36955 | Pigment epithelium-derived factor precursor | 2 | 1 |
| Q8TAA3 | Proteasome subunit alpha type 7-like | 2 | 1 |
| P21980 | Protein-glutamine gamma-glutamyltransferase | 2 | 1 |
| P00441 | Superoxide dismutase [Cu—Zn] | 2 | 1 |
| P37802 | Transgelin 2 | 2 | 1 |
| P00938 | Triosephosphate isomerase | 2 | 1 |
| P09493 | Tropomyosin 1 alpha chain | 3 | 1 |
| P06753 | Tropomyosin alpha 3 chain | 4 | 1 |
| P67936 | Tropomyosin alpha 4 chain | 5 | 1 |
| P05215 | Tubulin alpha-4 chain | 10 | 5 |
| Q99867 | Tubulin beta-4q chain | 2 | 1 |
| P38606 | Vacuolar ATP synthase catalytic subunit A, ubiquitous iso | 4 | 1 |
| P26640 | Valyl-tRNA synthetase | 2 | 1 |

Proteins identified in control and glaucomatous optic nerve

| | | | |
|---|---|---|---|
| P42655 | 14-3-3 protein epsilon | 6 | 3 |
| P61981 | 14-3-3 protein gamma | 2 | 3 |
| P27348 | 14-3-3 protein tau | 3 | 4 |
| P29312 | 14-3-3 protein zeta/delta | 4 | 5 |
| P09543 | 2′,3′-cyclic-nucleotide 3′-phosphodiesterase | 9 | 12 |
| O94811 | 25 kDa brain-specific protein | 2 | 2 |
| P11021 | 78 kDa glucose-regulated protein precursor | 2 | 3 |
| Q99798 | Aconitate hydratase, mitochondrial precursor | 4 | 3 |
| P02571 | Actin | 3 | 4 |
| P60709 | Actin, cytoplasmic 1 | 11 | 2 |
| P63261 | Actin, cytoplasmic 2 | 4 | 3 |
| P02511 | Alpha crystallin B chain | 5 | 11 |
| P06733 | Alpha-enolase | 9 | 13 |
| P01009 | Alpha-1-antitrypsin precursor | 4 | 11 |
| P12814 | Alpha-actinin 1 | 3 | 4 |
| O43707 | Alpha-actinin 4 | 4 | 5 |
| Q16352 | Alpha-internexin | 2 | 3 |
| P04083 | Annexin A1 | 2 | 8 |
| P07355 | Annexin A2 | 9 | 11 |
| P08758 | Annexin A5 | 6 | 12 |
| P08133 | Annexin A6 | 4 | 9 |
| P02647 | Apolipoprotein A-I precursor | 4 | 4 |
| P25705 | ATP synthase alpha chain, mitochondrial | 2 | 3 |
| P06576 | ATP synthase beta chain, mitochondrial precursor | 13 | 7 |
| P13929 | Beta enolase | 4 | 9 |
| P21810 | Biglycan precursor | 2 | 4 |
| P06702 | Calgranulin B | 2 | 4 |
| P16152 | Carbonyl reductase [NADPH] 1 | 6 | 11 |
| P18582 | CD81 antigen | 2 | 2 |
| P21926 | CD9 antigen | 3 | 5 |
| P60953 | Cell division control protein 42 homolog | 2 | 4 |
| P00450 | Ceruloplasmin precursor | 3 | 3 |
| Q00610 | Clathrin heavy chain 1 | 5 | 6 |
| P23528 | Cofilin, non-muscle isoform | 2 | 8 |
| P12109 | Collagen alpha 1 | 4 | 7 |
| P12277 | Creatine kinase, B chain | 6 | 12 |
| P07585 | Decorin precursor | 5 | 9 |
| Q14194 | Dihydropyrimidinase related protein-1 | 2 | 4 |
| Q16555 | Dihydropyrimidinase related protein-2 | 11 | 8 |
| Q14195 | Dihydropyrimidinase related protein-3 | 2 | 7 |
| P14625 | Endoplasmin precursor | 6 | 3 |
| P02794 | Ferritin heavy chain | 2 | 3 |
| P02792 | Ferritin light chain | 2 | 5 |
| Q06828 | Fibromodulin precursor | 2 | 2 |
| P09382 | Galectin-1 | 3 | 5 |
| P09104 | Gamma enolase | 5 | 10 |
| P06396 | Gelsolin precursor, plasma | 12 | 9 |
| P14136 | Glial fibrillary acidic protein, astrocyte | 16 | 13 |
| P06744 | Glucose-6-phosphate isomerase | 3 | 6 |
| P09211 | Glutathione S-transferase P | 3 | 9 |
| P00354 | Glyceraldehyde-3-phosphate dehydrogenase | 11 | 8 |
| P04406 | Glyceraldehyde-3-phosphate dehydrogenase | 3 | 12 |
| P11216 | Glycogen phosphorylase, brain form | 2 | 6 |
| P04901 | Guanine nucleotide-binding protein G | 2 | 7 |
| P08107 | Heat shock 70 kDa protein 1 | 4 | 4 |
| O43301 | Heat shock 70 kDa protein 12A | 2 | 3 |
| P17066 | Heat shock 70 kDa protein 6 | 2 | 3 |
| P11142 | Heat shock cognate 71 kDa protein | 4 | 4 |
| P07900 | Heat shock protein HSP 90-alpha | 7 | 4 |
| P08238 | Heat shock protein HSP 90-beta | 8 | 2 |
| P54652 | Heat shock-related 70 kDa protein 2 | 4 | 3 |
| P01922 | Hemoglobin alpha chain | 2 | 5 |
| P02790 | Hemopexin precursor | 2 | 3 |
| P02261 | Histone H2A.c/d/i/n/p | 2 | 2 |
| P62807 | Histone H2B.a/g/h/k/l | 2 | 3 |
| P68431 | Histone H3.1 | 2 | 2 |
| P62805 | Histone H4 | 5 | 4 |
| P01857 | Ig gamma-1 chain C region | 6 | 10 |
| P01859 | Ig gamma-2 chain C region | 5 | 7 |
| P01861 | Ig gamma-4 chain C region | 3 | 4 |
| P01834 | Ig kappa chain C region | 3 | 9 |
| P10745 | Interphotoreceptor retinoid-binding protein precursor | 12 | 3 |
| P13645 | Keratin, type I cytoskeletal 10 | 17 | 14 |
| P02533 | Keratin, type I cytoskeletal 14 | 9 | 7 |
| P08779 | Keratin, type I cytoskeletal 16 | 8 | 6 |
| P35527 | Keratin, type I cytoskeletal 9 | 14 | 11 |
| P04264 | Keratin, type II cytoskeletal 1 | 13 | 14 |
| P35908 | Keratin, type II cytoskeletal 2 epidermal | 6 | 14 |
| P19013 | Keratin, type II cytoskeletal 4 | 2 | 7 |
| P13647 | Keratin, type II cytoskeletal 5 | 6 | 6 |
| P02538 | Keratin, type II cytoskeletal 6A | 8 | 8 |
| P04259 | Keratin, type II cytoskeletal 6B | 2 | 2 |
| P48669 | Keratin, type II cytoskeletal 6F | 8 | 6 |
| P08729 | Keratin, type II cytoskeletal 7 | 3 | 2 |
| P00338 | L-lactate dehydrogenase A chain | 4 | 4 |
| P07195 | L-lactate dehydrogenase B chain | 6 | 11 |
| P51884 | Lumican precursor | 5 | 10 |
| P61626 | Lysozyme C | 2 | 3 |
| Q14764 | Major vault protein | 3 | 2 |
| P40925 | Malate dehydrogenase, cytoplasmic | 3 | 5 |
| P20774 | Mimecan precursor | 2 | 8 |
| P26038 | Moesin | 3 | 5 |
| P02686 | Myelin basic protein | 4 | 11 |
| P25189 | Myelin P0 protein (MPP) | 2 | 3 |
| P60201 | Myelin proteolipid protein (PLP) | 4 | 8 |
| P20916 | Myelin-associated glycoprotein precursor | 3 | 8 |
| Q16653 | Myetin-oligodendrocyte glycoprotein precursor | 2 | 2 |
| Q8IXJ6 | NAD-dependent deacetylase sirtuin 2 | 2 | 5 |
| P12036 | Neurofilament triplet H protein | 3 | 6 |
| P07196 | Neurofilament triplet L protein | 2 | 4 |
| P07197 | Neurofilament triplet M protein | 3 | 2 |
| P05092 | Peptidyl-prolyl cis-trans isomerase A | 2 | 2 |
| P62942 | Peptidyl-prolyl cis-trans isomerase A | 2 | 2 |
| Q06830 | Peroxiredoxin 1 | 3 | 6 |
| P32119 | Peroxiredoxin 2 | 3 | 7 |
| P30041 | Peroxiredoxin 6 | 2 | 10 |
| P30086 | Phosphatidylethanolamine-binding protein | 2 | 10 |
| P00558 | Phosphoglycerate kinase 1 | 3 | 5 |
| P18669 | Phosphoglycerate mutase 1 | 3 | 6 |
| P07737 | Profilin I | 2 | 7 |
| P51888 | Prolargin precursor | 3 | 6 |
| P11498 | Pyruvate carboxylase mitochondrial precursor | 3 | 3 |
| P14618 | Pyruvate kinase, isozymes M1/M2 | 14 | 13 |
| P31150 | Rab GDP dissociation inhibitor alpha | 3 | 3 |
| P35241 | Radixin | 2 | 2 |
| P04271 | S-100 protein, beta chain | 2 | 5 |
| Q13228 | Selenium-binding protein 1 | 3 | 2 |
| Q15019 | Septin 2 | 3 | 8 |
| Q16181 | Septin 7 | 2 | 7 |
| P02787 | Serotransferrin precursor | 13 | 11 |
| P02768 | Serum albumin precursor | 35 | 13 |
| P05023 | Sodium/potassium-transporting ATPase alpha-1 chain pre | 6 | 6 |
| P50993 | Sodium/potassium-transporting ATPase alpha-2 chain pre | 8 | 6 |
| P13637 | Sodium/potassium-transporting ATPase alpha-3 chain | 6 | 3 |
| P05026 | Sodium/potassium-transporting ATPase beta-1 chain | 3 | 2 |
| Q13813 | Spectrin alpha chain, brain | 4 | 8 |
| Q01082 | Spectrin beta chain, brain 1 | 11 | 7 |
| P04179 | Superoxide dismutase [Mnj, mitochondrial precursor | 3 | 3 |
| Q01995 | Transgelin | 2 | 3 |
| P55072 | Transitional endoplasmic reticulum ATPase | 5 | 7 |
| P29401 | Transketolase | 4 | 7 |
| P40939 | Trifunctional enzyme alpha subunit, mitochondrial precurs | 3 | 2 |
| P60174 | Triosephosphate isomerase | 3 | 8 |
| P05209 | Tubulin alpha-1 chain | 10 | 12 |
| P68366 | Tubulin alpha-1 chain | 7 | 3 |
| Q13748 | Tubulin alpha-2 chain | 10 | 3 |
| P05215 | Tubulin alpha-4 chain | 4 | 9 |
| Q9BQE3 | Tubulin alpha-6 chain | 5 | 2 |
| P07437 | Tubulin beta-1 chain | 12 | 6 |
| P05217 | Tubulin beta-2 chain | 10 | 8 |
| Q13509 | Tubulin beta-4 chain | 3 | 2 |
| P05218 | Tubulin beta-5 chain | 13 | 12 |
| P04350 | Tubulin beta-5 chain | 5 | 8 |
| P62988 | Ubiquitin | 2 | 5 |
| Q9UHP3 | Ubiquitin carboxyl-terminal hydrolase 25 | 2 | 2 |
| P09936 | Ubiquitin carboxyl-terminal hydrolase isozyme L1 | 3 | 3 |
| P22314 | Ubiquitin-activating enzyme E1 | 6 | 8 |
| P08670 | Vimentin | 8 | 14 |
| P18206 | Vinculin | 10 | 3 |
| P21796 | Voltage-dependent anion-selective channel protein 1 | 2 | 6 |

^{a}Swiss-Protein database accession numbers are shown (http://us.expasy.org/sprot/)
^{b}Number of donors exhibiting the indicated protein

TABLE 2
Anti-citrulline IP Products identified by LC MS/MS

| Accession | | Peptide | |
|---|---|---|---|
| Number^{a} | Protein | Matches | M_{calc.} |
| P62258 | 14-3-3 protein epsilon | 6 | 29155 |
| P61981 | 14-3-3 protein gamma | 3 | 28171 |
| P63104 | 14-3-3 protein zeta/delta | 3 | 27727 |
| P09543 | 2′,3′-cyclic-nucleotide 3′-phosphodiesterase | 10 | 47560 |
| P11021 | 78 kDa glucose-regulated protein | 5 | 72315 |
| O43707 | Alpha-actinin 4 | 7 | 104836 |
| P04083 | Annexin A1 | 6 | 38565 |
| P07355 | Annexin A2 | 15 | 38454 |
| P08758 | Annexin A5 | 8 | 35787 |
| P08133 | Annexin A6 | 6 | 75724 |
| P07585 | Decorin | 5 | 39728 |
| P09417 | Dihydropteridine reductase | 2 | 25785 |
| Q14194 | Dihydropyrimidinase related protein-1 | 3 | 62165 |
| Q16555 | Dihydropyrimidinase related protein-2 | 10 | 62275 |
| Q14195 | Dihydropyrimidinase related protein-3 | 3 | 61945 |
| P09104 | Gamma enolase | 7 | 47119 |
| P06396 | Gelsolin, plasma | 5 | 85679 |
| P14136 | Glial fibriliary acidic protein, astrocyte | 13 | 49862 |
| P62805 | Histone H4 | 5 | 11236 |
| P51884 | Lumican | 4 | 38411 |
| P20774 | Mimecan | 3 | 33904 |
| P02686 | Myelin basic protein | 1^{b} | 33099 |
| P25189 | Myelin P0 protein | 2 | 27555 |
| P60201 | Myelin proteolipid protein 1 | 1^{c} | 29946 |
| P20916 | Myelin-associated glycoprotein | 1^{d} | 69050 |
| Q16653 | Myelin-oligodendrocyte glycoprotein | 3 | 28179 |
| P13591 | Neural cell adhesion molecule (N-CAM 140) | 1^{e} | 93360 |
| P12036 | Neurofilament triplet H protein | 2 | 112461 |
| P51888 | Prolargin | 3 | 43791 |
| Q15019 | Septin 2 | 4 | 41459 |
| Q16181 | Septin 7 | 2 | 48769 |
| P02787 | Serotransferrin | 9 | 77032 |
| P55072 | Transitional endoplasmic reticulum ATPase | 7 | 89172 |
| Q99867 | Tubulin beta-4q chain | 11 | 48434 |
| P05218 | Tubulin beta-5 chain | 7 | 49652 |
| P21796 | Voltage-dependent anion-selective channel protein | 3 | 30623 |

^{a}Swiss-Protein database accession numbers are shown (http://us.expasy.org/sprot/).
^{b}The identified peptide and determined sequence (underlined) for myelin basic protein: RHRDTGILDSIGRF.
^{c}The identified peptide and determined sequence (underlined) for myelin proteoiipid protein 1: MYGVLPWNAFPGK.
^{d}The identified peptide and determined sequence (underlined) for myelin-assoicated glycoprotein: RSGLVLTSILTLRG.
^{e}The identified peptide

| | | | | | | | |
|---|---|---|---|---|---|---|---|
| | | | | | | | *Glaucoma |
| Age | Gender | Race | *PMI | Cause of Death | Medications | *C/D | Scaling |

Glaucomatous Donors

| | | | | | | | | |
|---|---|---|---|---|---|---|---|---|
| 75 | F | Caucasian | 3.5 | h | Heart attack | Lasix, Lescol, | 0.7 | +++ |
| | | | | | | Lovenox, Lescol, | | |
| | | | | | | Metrazolamide | | |
| 73 | F | Caucasian | 3 | h | Respiratory Failure | Tylenol, KCl, | 0.6 | +++ |
| | | | | | | lidocaine, Lasix, | | |
| | | | | | | KCl, Xalatan | | |
| 76 | F | Caucasian | 3 | h | Acute Myocardial | Digoxin, TYlenol, | 0.7 | +++ |
| | | | | | Infarction | ASA, Esmolol, | | |
| | | | | | | Colace, Xalatan | | |
| 79 | M | Caucasian | 3.5 | h | Acute Myocardial | Coomadin, | 1 | +++ |
| | | | | | Infarction | Digoxin, Ilowex, | | |
| | | | | | | ASA, Cosopt, epi, | | |
| | | | | | | Esmolol, Colace, | | |
| | | | | | | Iopressor | | |
| 55 | M | Caucasian | 6 | h | Heart trauma, | Pepcid, Heparin, | 0.7 | +++ |
| | | | | | Hyperlipidemia, | Hydrocortisone, | | |
| | | | | | respiratory failure | Ativa, levophed, | | |
| | | | | | | Xalatan and | | |
| | | | | | | Drotrecogin | | |
| 72 | F | Caucasian | 3.5 | h | Respiratory failure, | Tylenol, Ambien, | 0.7 | +++ |
| | | | | | gallbladder | KCl, Atropine, | | |
| | | | | | removal | Ativa, lidocain, | | |
| | | | | | | Zotran | | |
| 58 | M | Caucasian | 4 | h | Cardiac arrest | Xalatan, Betimol | 0.6 | +++ |
| 86 | F | Caucasian | 5 | h | Respiratory Arrest, | Tylenol, Ambien, | 0.6 | ++ |
| | | | | | Ovarian cancer | Atropine, Ativan, | | |
| | | | | | | lidocaine, Xalatan | | |
| 81 | F | Caucasian | 4 | h | Cardiac arrest, | Zemotron, | 0.5 | ++ |
| | | | | | Arthritis, | Lumigan, Lescol, | | |
| | | | | | hyperlipidemia, | KCl, mannitol, ASA, | | |
| | | | | | hypertension | Lovenox, Lescol, | | |
| | | | | | | Timolol, Xalatan | | |
| 86 | M | Caucasian | 3.5 | h | Respiratory arrest, | Tylenol, KCl, | 0.6 | +++ |
| | | | | | Osteoporosis | Atropine, Ativan, | | |
| | | | | | | lidocaine, Xalatan | | |
| 84 | M | Caucasian | 4 | h | Lung and Colon | Tylenol, Ambien, | 0.8 | +++ |
| | | | | | Cancer, Jundice, | Roxanal, Ativan, | | |
| | | | | | Liver problems | Lovanox, Lasix, | | |
| | | | | | | Potassium | | |
| 85 | M | Caucasian | 4 | h | Heart attack | Lasix, Tylenol, KCl, | 0.7 | +++ |
| | | | | | | Lovenox | | |

Control Donors

| | | | | | | | |
|---|---|---|---|---|---|---|---|
| 82 | M | Caucasian | 3.5 | h | Sudden cardiac | Lasix, Ativan, | 0.4 |
| | | | | | failure | Tylenol, | |
| | | | | | | Dobutamine, KCl | |
| 80 | F | Caucasian | 5 | h | Respiratory arrest | Tylenol, Ativan, | 0.4 |
| | | | | | | lidocaine | |
| 72 | F | Caucasian | 5 | h | Hypothyroidism, | Lasix, Dobutamine, | N/A |
| | | | | | Heart Attack | CaCl, Ativan | |
| 55 | M | Caucasian | 5.5 | h | Heart attack, renal | Zusyn, | N/A |
| | | | | | failure | Gentamycin, | |
| | | | | | | pepcid, Ativan, | |
| | | | | | | Levophed, | |
| | | | | | | Vancomycin | |
| 67 | F | Caucasian | 5 | h | Heart attack | Pepcid, Tylenol, | 0.4 |
| | | | | | | Ativan | |
| 73 | F | Caucasian | 3.5 | h | Lung Cancer, | Tussionex, celtriaxone, | N/A |
| | | | | | Adrenal problem | decadron, albuterol, | |
| | | | | | | ipratropium, lorazopam, | |
| | | | | | | morphine | |
| 63 | M | Caucasian | 6 | h | Fibromyalgia, | Pepcid, heparin, | 0.4 |
| | | | | | heart attack | Hydrocortisone, | |
| | | | | | | Ativan, levophed | |
| 85 | M | Caucasian | 3.5 | h | Nephropathy, | Vancomycin, | N/A |
| | | | | | Cardiac arrest | Levophed, Lescol, | |
| | | | | | | Tylenol | |
| 82 | F | Caucasian | 6 | h | Ovarian Cancer, | Lasix, Lescol, KCl, | N/A |
| | | | | | Cardiac arrest | Lovenox, Lescol | |
| 87 | M | Caucasian | 4.5 | h | Cardiac arrest | Lasix, KCl, Lescol, | N/A |
| | | | | | | Tylenol | |
| 87 | M | Caucasian | 4.5 | h | Cardiac arrest | Tylenol, KCl, | 0.4 |
| | | | | | | Ativan, Lovenox, | |
| | | | | | | Lescol | |
| 77 | F | Caucasian | 3 | h | Sudden cardiac | Celtriaxone, | N/A |
| | | | | | arrest | decadron, Lasix, | |
| | | | | | | albuterol | |

*PMI = Post mortem to enucleation time. C/D = Cup to disc ratio. Glaucoma scaling ++ Moderate; +++ Severe/Progressed glaucoma. Glaucoma scaling is based on a static perimetry threshold test (30-2), glaucomatous hemifield test and mean field defect (MD) where MD mild = 0 to −2, MD moderate = −2 to −10 and MD severe is greater than −10.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inhibiting optic nerve damage in an individual in need thereof, comprising administering to the individual an agent that inhibits peptidyl arginine deiminase 2 (PAD2).

2. The method of claim 2 wherein the agent inhibits expression of PAD2, biological activity of PAD2 or a combination thereof.

3. The method of claim 2 wherein the agent directly inhibits the expression of PAD2.

4. The method of claim 3 wherein the agent is interfering RNA.

5. The method of claim 2 wherein the biological activity of PAD2 that is inhibited is increased protein citrullination, decreased protein arginyl methylation or a combination thereof.

6. The method of claim 5 wherein protein citrullination of at least one optic nerve protein is inhibited.

7. The method of claim 6 wherein the optic nerve protein is a myelin protein.

8. The method of claim 7 wherein the myelin protein is selected from the group consisting of: myelin basic protein, myelin proteolipid protein, myelin associated glycoprotein, myelin P0 protein, myelin oligodendrocyte protein and a combination thereof.

9. A method of inhibiting glaucomatous optic nerve damage in an individual in need thereof, comprising administering to the individual an agent that inhibits peptidyl arginine deiminase 2 (PAD2).

10. A method of treating glaucoma in an individual in need thereof, comprising administering to the individual an agent that specifically inhibits peptidyl arginine deiminase 2 (PAD2).

11. The method of claim 10 wherein the glaucoma is primary open angle glaucoma.

12. A method of identifying an agent that can be used to inhibit optic nerve damage comprising:
a) contacting a cell or animal which expresses peptidyl arginine deiminase 2 (PAD2) with an agent to be assessed;
b) assessing the level of expression or biological activity of PAD2 in the cell of animal,
wherein if the level of expression or biological activity of PAD2 is decreased in the presence of the agent, then the agent can be used to inhibit optic nerve damage.

13. The method of claim 12 wherein the cell is an ocular cell.

14. The method of claim 13 wherein the ocular cell is an astrocyte.

15. The method of claim 12 wherein the animal is an animal model of glaucoma.

16. The method of claim 14 wherein the animal model is a DBA/2J mouse.

17. The method of claim 12 wherein the biological activity of PAD2 that is assessed is protein citrullination and if protein citrullination is decreased, then the agent can be used to inhibit optic nerve damage.

18. The method of claim 16 wherein protein citrullination of at least one optic nerve protein is assessed.

19. The method of claim 18 wherein the optic nerve proteins is a myelin protein.

20. The method of claim 19 wherein the myelin protein is selected from the group consisting of: myelin basic protein, myelin proteolipid protein, myelin associated glycoprotein, myelin P0 protein, myelin oligodendrocyte protein and a combination thereof.

21. The method of claim 12 wherein the biological activity of PAD2 that is assessed is citrullination and if citrullination is increased, then the agent can be used to inhibit optic nerve damage.

22. The method of claim 12 wherein the agent can be used to treat optic nerve damage.

23. The method of claim 12 wherein the agent can be used to treat glaucoma.

24. The method of claim 23 wherein the glaucoma is primary open angle glaucoma.


SEQUENCE LISTING (21 sequences)

SEQ ID NO: 1 - DNA, 20 bp, Unknown
  Probe for PAD2
aaacctggaggtcagtcccc

SEQ ID NO: 2 - DNA, 20 bp, Unknown
  Probe for PAD2
aaacctggaggtcagtcccc

SEQ ID NO: 3 - DNA, 21 bp, Unknown
  Probe for GPDH
cttcaccaccatggagaaggc

SEQ ID NO: 4 - DNA, 21 bp, Unknown
  Probe for GPDH
ggcatggactgtggtcatgag

SEQ ID NO: 5 - DNA, 24 bp, Unknown
  Probe for HGRT
gaagagctactgtaatgatcagtc

SEQ ID NO: 6 - DNA, 23 bp, Unknown
  Probe for HGRT
aaagtctggcctgtatccaacac

SEQ ID NO: 7 - DNA, 97 bp, Unknown
  shRNA sequence against PAD2
tgctgttgacagtgagcgacagccttgactcatttggaaatagtgaagccacagatgtatttccaaatgagtcaaggctggtgcctactgcctcgga

SEQ ID NO: 8 - DNA, 25 bp, Unknown
  Portion of PAD2 DNA sequence for design of shRNA
ggatacgagatagttctgtacattt

SEQ ID NO: 9 - DNA, 25 bp, Unknown
  Portion of PAD2 DNA sequence for design of shRNA
cccatcttcacggacaccgtgatat

SEQ ID NO: 10 - DNA, 25 bp, Unknown
  Portion of PAD2 DNA sequence for design of shRNA
cccgagatggaaacctaaaggactt

SEQ ID NO: 11 - DNA, 25 bp, Unknown
  Portion of PAD2 DNA sequence for design of shRNA
ggatgagcagcaagcgaatcaccat

SEQ ID NO: 12 - DNA, 25 bp, Unknown
  Portion of PAD2 DNA sequence for design of shRNA
gccttcttcccaaacatggtgaaca

SEQ ID NO: 13 - DNA, 21 bp, Unknown
  Portion of PAD2 sequence (NM 007365) for design of siRNA
ggucaccgucaacuacuautt

SEQ ID NO: 14 - DNA, 21 bp, Unknown
  Portion of PAD2 sequence (NM 007365) for design of siRNA
auaguaguugacggugacctt

SEQ ID NO: 15 - DNA, 21 bp, Unknown
  Portion of PAD2 sequence (NM 007365) for design of siRNA
gaacaacccaaagaaggcatt

SEQ ID NO: 16 - DNA, 21 bp, Unknown
  Portion of PAD2 sequence (NM 007365) for design of siRNA
ugccuucuuuggguuguuctt

SEQ ID NO: 17 - DNA, 21 bp, Unknown
  Portion of PAD2 sequence (NM 007365) for design of siRNA
cgcuauauccacauccuggtt

SEQ ID NO: 18 - DNA, 21 bp, Unknown
  Portion of PAD2 sequence (NM 007365) for design of siRNA
ccaggauguggauauagcgtt

SEQ ID NO: 19 - DNA, 97 bp, Unknown
  shRNA in pShag-magic 2 (coding region of PAD2)
tccgaggcagtaggcaccagccttgactcatttggaaatacatctgtggcttcactatttccaaatgagtcaaggctgtcgctcactgtcaacagca

SEQ ID NO: 20 - DNA, 2348 bp, Unknown
  nucleotide sequence of human PAD2 (NM-007365)
cgcacctgctgcaggtgctcccggccgccccggaccagcgagcgcgggcactgcggcggggaggatgctgcgcgagcggaccgtgcggctgcagtacgggagccgcgtggaggcggtgtacgtgctgggcacctacctctggaccgatgtctacagcgcggccccagccggggcccaaaccttcagcctgaagcactcggaacacgtgtgggtggaggtggtgcgtgatggggaggctgaggaggtggccaccaatggcaagcagcgctggcttctctcgcccagcaccaccctgcgggtcaccatgagccaggcgagcaccgaggccagcagtgacaaggtcaccgtcaactactatgacgaggaagggagcattcccatcgaccaggcggggctcttcctcacagccattgagatctccctggatgtggacgcagaccgggatggtgtggtggagaagaacaacccaaagaaggcatcctggacctggggccccgagggccagggggccatcctgctggtgaactgtgaccgagagacaccctggttgcccaaggaggactgccgtgatgagaaggtctacagcaaggaagatctcaaggacatgtcccagatgatcctgcggaccaaaggccccgaccgcctccccgccggatacgagatagttctgtacatttccatgtcagactcagacaaagtgggcgtgttctacgtggagaacccgttcttcggccaacgctatatccacatcctgggccggcggaagctctaccatgtggtcaagtacacgggtggctccgcggagctgctgttcttcgtggaaggcctctgtttccccgacgagggcttctcaggcctggtctccatccatgtcagcctgctggagtacatggcccaggacattcccctgactcccatcttcacggacaccgtgatattccggattgctccgtggatcatgacccccaacatcctgcctcccgtgtcggtgtttgtgtgctgcatgaaggataattacctgttcctgaaagaggtgaagaaccttgtggagaaaaccaactgtgagctgaaggtctgcttccagtacctaaaccgaggcgatcgctggatccaggatgaaattgagtttggctacatcgaggccccccataaaggcttccccgtggtgctggactctccccgagatggaaacctaaaggacttccctgtgaaggagctcctgggcccagattttggctacgtgacccgggagcccctctttgagtctgtcaccagccttgactcatttggaaacctggaggtcagtcccccagtgaccgtgaacggcaagacatacccgcttggccgcatcctcatcgggagcagctttcctctgtctggtggtcggaggatgaccaaggtggtgcgtgacttcctgaaggcccagcaggtgcaggcacccgtggagctctactcagactggctgactgtgggccacgtggatgagttcatgtcctttgtccccatccccggcacaaagaaattcctgctactcatggccagcacctcggcctgctacaagctcttccgagagaagcagaaggacggccatggagaggccatcatgttcaaaggcttgggtgggatgagcagcaagcgaatcaccatcaacaagattctgtccaacgagagccttgtgcaggagaacctgtacttccagcgctgcctggactggaaccgtgacatcctcaagaaggagctgggactgacagagcaggacatcattgacctgcccgctctgttcaagatggacgaggaccaccgtgccagagccttcttcccaaacatggtgaacatgatcgtgctggacaaggacctgggcatccccaagccattcgggccacaggttgaggaggaatgctgcctggagatgcacgtgcgtggcctcctggagcccctgggcctcgaatgcaccttcatcgacgacatttctgcctaccacaaatttctgggggaagtccactgtggcaccaacgtccgcaggaagcccttcaccttcaagtggttgcacatggtgccctgacctgccaggggccctggcgtttgcctccttcgcttagttctccagaccctccctcacacgcccagagccttctgctgacatggactggacagccccgctgggagacctttgggacgtggggtggaatttggggtatctgtgccttgccctccctgagaggggcctcagtgtcctctgaagccatccccagtgagcctcgactctgtccctgctgaaaatagctgggccagtgtctctgtagccctgacataaggaacagaacacaacaaaacacagcaaaccatgt

SEQ ID NO: 21 - protein, 665 aa, Unknown
  amino acid sequence of human PAD2 (NM-007365)
MLRERTVRLQYGSRVEAVYVLGTYLWTDVYSAAPAGAQTFSLKHSEHVWVEVVRDGEAEEVATNGKQRWLLSPSTTLRVTMSQASTEASSDKVTVNYYDEEGSIPIDQAGLFLTAIEISLDVDADRDGVVEKNNPKKASWTWGPEGQGAILLVNCDRETPWLPKEDCRDEKVYSKEDLKDMSQMILRTKGPDRLPAGYEIVLYISMSDSDKVGVFYVENPFFGQRYIHILGRRKLYHVVKYTGGSAELLFFVEGLCFPDEGFSGLVSIHVSLLEYMAQDIPLTPIFTDTVIFRIAPWIMTPNILPPVSVFVCCMKDNYLFLKEVKNLVEKTNCELKVCFQYLNRGDRWIQDEIEFGYIEAPHKGFPVVLDSPRDGNLKDFPVKELLGPDFGYVTREPLFESVTSLDSFGNLEVSPPVTVNGKTYPLGRILIGSSFPLSGGRRMTKVVRDFLKAQQVQAPVELYSDWLTVGHVDEFMSFVPIPGTKKFLLLMASTSACYKLFREKQKDGHGEAIMFKGLGGMSSKRITINKILSNESLVQENLYFQRCLDWNRDILKKELGLTEQDIIDLPALFKMDEDHRARAFFPNMVNMIVLDKDLGIPKPFGPQVEEECCLEMHVRGLLEPLGLECTFIDDISAYHKFLGEVHCGTNVRRKPFTFKWLHMVP